(19) United States
(12) Patent Application Publication
(10) Pub. No.: US 2011/0124690 A1
(43) Pub. Date: May 26, 2011

(54) COMPOSITIONS AND METHODS FOR TREATING CANCER OR A NEUROTROPHIC DISORDER

(76) Inventors: Samuel J. Danishefsky, Englewood, NJ (US); Heedong Yun, Tenafly, NJ (US); Ting-Chao Chou, Paramus, NJ (US); Xiaoguang Lei, Beijing (CN); Dalibor Sames, New York, NY (US)

(21) Appl. No.: 12/528,415

(22) PCT Filed: Feb. 22, 2008
(86) PCT No.: PCT/US08/54737

§ 371 Date: Feb. 10, 2011

Related U.S. Application Data
(60) Provisional application No. 60/903,175, filed on Feb. 23, 2007.

(51) Int. Cl. A61K 31/357; C07D 317/20; A61K 31/427; A61K 31/047; C07C 49/24; A61K 31/121; C07D 317/26; C07C 45/29; C07D 317/30; A61P 35/00; A61P 25/00

(52) U.S. Cl. 514/365; 549/453; 514/467; 514/738; 568/415; 514/675; 549/454; 568/405

(57) ABSTRACT

The present invention relates to compositions comprising an effective amount of a Panaxytriol Compound and a tubulin-binding drug, methods for treating or preventing cancer or a neurotrophic disorder comprising administering to a subject in need thereof an effective amount of a Panaxytriol Compound and a tubulin-binding drug, and methods for making a Panaxytriol Compound.

### 1. FIELD OF THE INVENTION

The present invention relates to compositions comprising an effective amount of a Panaxytriol Compound and a tubulin-binding drug, methods for treating or preventing cancer or a neurotrophic disorder comprising administering to a subject in need thereof an effective amount of a Panaxytriol Compound and a tubulin-binding drug, and methods for making a Panaxytriol Compound.

### 2. BACKGROUND OF THE INVENTION

Cancer is second only to cardiovascular disease as the leading cause of death in the United States. The American Cancer Society estimated that 1.4 million new cancer cases would be diagnosed and 565,000 people would die of cancer in 2006 (American Cancer Society, Cancer Facts and Figures 2006, Atlanta, Ga.). The National Cancer Institute estimated that in January 2002, approximately 10.1 million living Americans had a history of cancer. The National Institutes of Health estimate direct medical costs of cancer as over $100 billion per year with an additional $100 billion in indirect costs due to lost productivity—the largest such costs of any major disease.

Cancer is a process by which the controlling mechanisms that regulate cell growth and differentiation are impaired, resulting in a failure to control cell turnover and growth. This lack of control can cause a tumor to grow progressively, enlarging and occupying space in vital areas of the body. If the tumor invades surrounding tissue and is transported to distant sites, death of the individual can result.

The selective killing of cancer cells, while minimizing deleterious effects on normal cells, is a desired goal in cancer therapy. Modalities commonly used in the treatment of cancer include chemotherapy, radiation therapy, surgery and biological therapy (a broad category that includes gene-, protein- or cell-based treatments and immunotherapy). Despite the availability of a variety of anticancer agents, traditional chemotherapy has drawbacks. Many anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy is effective for only a very limited number of cancers. Many chemotherapeutic drugs are anti-proliferative agents, acting at different stages of the cell cycle. Since it is difficult to predict the pattern of sensitivity of a neoplastic cell population to anticancer drugs, or the current stage of the cell cycle that a cell happens to be in, it is common to use multi-drug regimens in the treatment of cancer.

Despite the significant research efforts and resources that have been directed towards the development of novel anticancer agents and improved methods for treating cancer there remains a need in the art for novel compounds, compositions, or methods that are useful for treating cancer with improved therapeutic indices.

Citation of any reference in Section 2 of this application is not an admission that the reference is prior art.

### 3. SUMMARY OF THE INVENTION

In one aspect the invention provides methods for treating or preventing cancer, comprising administering to a subject in need thereof and effective amount of a tubulin-binding drug and panaxytriol.

In another aspect, the invention provides methods for treating or preventing cancer, comprising administering to a subject in need thereof and effective amount of a tubulin-binding drug and Compound (A):

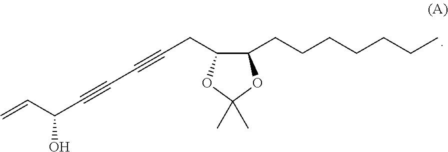

In yet another aspect, the invention provides methods for treating or preventing cancer, comprising administering to a subject in need thereof and effective amount of a tubulin-binding drug and Compound (B):

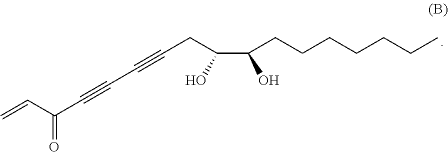

In still another aspect, the invention provides methods for treating or preventing cancer, comprising administering to a subject in need thereof and effective amount of a tubulin-binding drug and Compound (C):

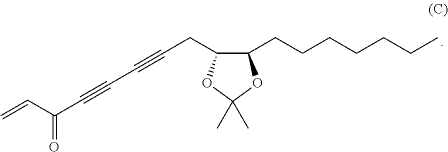

In still another aspect, the invention provides methods for treating or preventing cancer, comprising administering to a subject in need thereof and effective amount of a tubulin-binding drug and Compound (D):

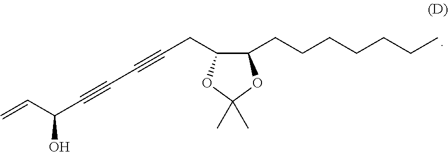

In still another aspect, the invention provides methods for treating or preventing cancer, comprising administering to a subject in need thereof and effective amount of a tubulin-binding drug and Compound (E):

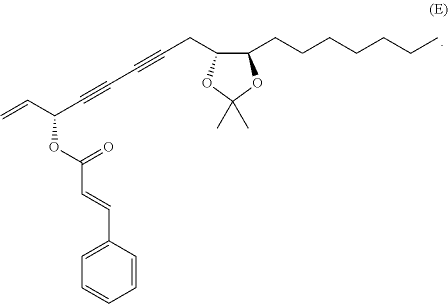

In still another aspect, the invention provides methods for treating or preventing cancer, comprising administering to a subject in need thereof and effective amount of a tubulin-binding drug and Compound (F):

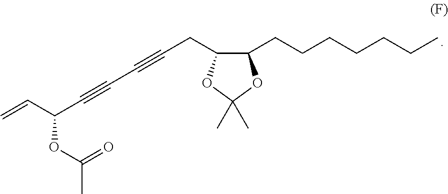

In one aspect the invention provides methods for treating or preventing a neurotrophic disorder, comprising administering to a subject in need thereof and effective amount of a tubulin-binding drug and panaxytriol.

In another aspect, the invention provides methods for treating or preventing a neurotrophic disorder, comprising administering to a subject in need thereof and effective amount of a tubulin-binding drug and Compound (A):

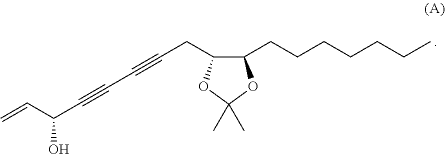

In yet another aspect, the invention provides methods for treating or preventing a neurotrophic disorder, comprising administering to a subject in need thereof and effective amount of a tubulin-binding drug and Compound (B):

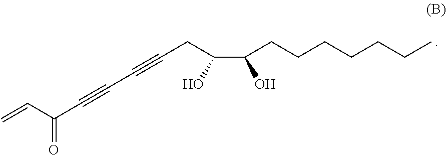

In still another aspect, the invention provides methods for treating or preventing a neurotrophic disorder, comprising administering to a subject in need thereof and effective amount of a tubulin-binding drug and Compound (C):

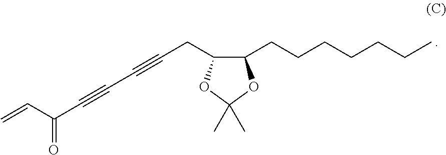

In still another aspect, the invention provides methods for treating or preventing a neurotrophic disorder, comprising administering to a subject in need thereof and effective amount of a tubulin-binding drug and Compound (D):

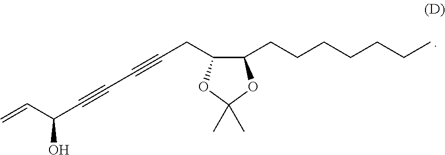

In still another aspect, the invention provides methods for treating or preventing a neurotrophic disorder, comprising administering to a subject in need thereof and effective amount of a tubulin-binding drug and Compound (E):

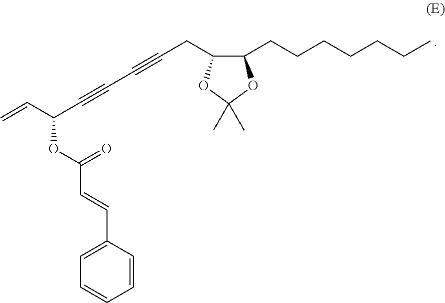

In still another aspect, the invention provides methods for treating or preventing a neurotrophic disorder, comprising administering to a subject in need thereof and effective amount of a tubulin-binding drug and Compound (F):

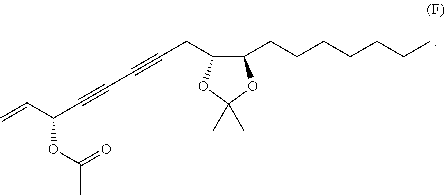

Methods comprising administering an effective amount of Panaxytriol Compound (A), Compound (B), Compound (C), Compound (D), Compound (E), or Compound (F) (a “Panaxytriol Compound”), and a tubulin-binding drug are useful for treating or preventing cancer or a neurotrophic disorder (each being a “Condition”).

The invention further provides compositions comprising a physiologically acceptable vehicle and an effective amount of a Panaxytriol Compound and a tubulin-binding drug. These compositions are useful for treating or preventing a condition.

In one aspect, the invention provides methods for making panaxytriol, comprising allowing the compound having the structure

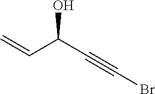

to react with the compound having the structure

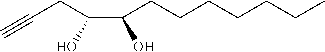

in the presence of CuCl and under conditions that are sufficient to make panaxytriol.

In another aspect, the invention provides a method for making Compound (A):

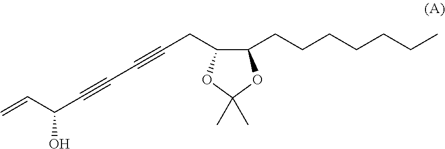

comprising allowing panaxytriol to react with 2,2-dimethoxypropane in the presence of a protic acid under conditions that are sufficient to make Compound (A). In one embodiment, the amount of the protic acid is a catalytic amount.

In yet another aspect, the invention provides a method for making Compound (B):

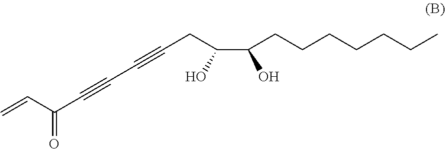

comprising oxidizing panaxytriol under conditions that are sufficient to make Compound (B).

In still another aspect, the invention provides a method for making Compound (C):

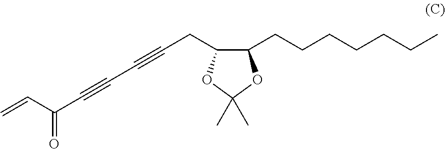

comprising oxidizing Compound (A):

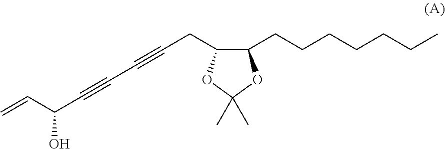

under conditions that are sufficient to make Compound (C).

In another aspect, the invention provides a method for making Compound (D):

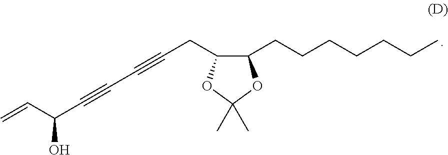

comprising allowing the compound having the structure

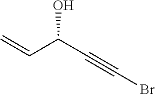

to react with the compound 6

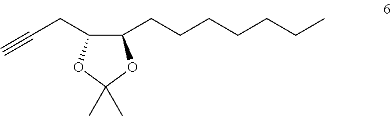

in the presence of CuCl and under conditions that are sufficient to make Compound (D).

In another aspect, the invention provides a method for making Compound (E):

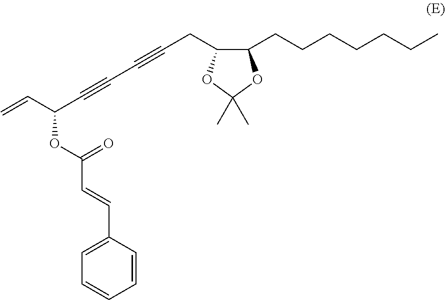

comprising allowing Compound (A) to react with cinnamic acid in the presence of a coupling agent under conditions that are sufficient to make Compound (E). In one embodiment, the coupling agent is DCC. In another embodiment, the conditions comprise a catalyst such as DMAP.

In another aspect, the invention provides a method for making Compound (F):

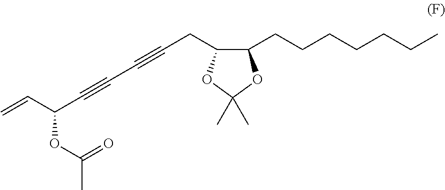

comprising allowing Compound (A) to react with acetic anhydride in the presence of a base under conditions that are sufficient to make Compound (F). In one embodiment, the coupling agent is DCC. In another embodiment, the base is pyridine, or a tertiary amine base such as triethylamine, or Hunig's base.

The details of the invention are set forth in the accompanying description below. All references cited in this specification are incorporated by reference in their entireties.

### 4. BRIEF DESCRIPTION OF THE DRAWINGS

FIG. 1 shows the therapeutic effect of panaxytriol in nude mice bearing MX-1 xenograft using various dosage regimens: ▴ represents a control, ▪ represents 30 mg/kg Q2D×3, 50 mg/kg Q2D×3 and 75 mg/kg Q2D×3; and represents 50 mg/kg Q2D×3, 75 mg/kg Q2D×3 and 100 mg/kg.

FIG. 2 shows the therapeutic effect of Compound (A) in nude mice bearing MX-1 xenograft using different dosage regimens: ▴ represents a control, ▪ represents 10 mg/kg Q2D×3, 30 mg/kg Q2D×3 and 50 mg/kg Q2D×3; and represents 20 mg/kg Q2D×3, 50 mg/kg Q2D×3 and 100 mg/kg; and

FIG. 3 shows images of neurite outgrowth with or without administration of panaxytriol.

### 5. DETAILED DESCRIPTION OF THE INVENTION

### 5.1 Definitions and Abbreviations

The following definitions are used in connection with the Panaxytriol Compounds:

A “tubulin-binding drug” refers to a ligand of tubulin or to a compound capable of binding α or β-tubulin monomers or oligomers thereof, αβ-tubulin heterodimers or oligomers thereof, or polymerized microtubules.

Illustrative tubulin-binding drugs include, but are not limited to:

a) Combretastatins or other stilbene analogs (Pettit et al, Can. J. Chem., 1982; Pettit et al, J. Org. Chem., 1985; Pettit et al, J. Nat. Prod., 1987; Lin et al, Biochemistry, 1989; Singh et al, J. Org. Chem., 1989; Cushman et al, J. Med. Chem., 1991; Getahun et al, J. Med. Chem., 1992; Andres et al, Bioorg. Med. Chem. Lett., 1993; Mannila, Liebigs. Ann. Chem., 1993; Shirai et al, Bioorg. Med. Chem. Lett., 1994; Medarde et al., Bioorg. Med. Chem. Lett., 1995; Pettit et al, J. Med. Chem., 1995; Wood et al, Br. J. Cancer., 1995; Bedford et al, Bioorg. Med. Chem. Lett., 1996; Dorr et al, Invest. New Drugs, 1996; Jonnalagadda et al., Bioorg. Med. Chem. Lett., 1996; Shirai et al, Heterocycles, 1997; Aleksandrzak K, Anticancer Drugs, 1998; Chen et al, Biochem. Pharmacol., 1998; Ducki et al, Bioorg. Med. Chem. Lett., 1998; Hatanaka et al, Bioorg. Med. Chem. Lett., 1998; Medarde, Eur. J. Med. Chem., 1998; Medina et al, Bioorg. Med. Chem. Lett., 1998; Ohsumi et al, Bioorg. Med. Chem. Lett., 1998; Ohsumi et al., J. Med. Chem., 1998; Pettit G R et al., J. Med. Chem., 1998; Shirai et al, Bioorg. Med. Chem. Left., 1998; Banwell et al, Aust. J. Chem., 1999; Medarde et al, Bioorg. Med. Chem. Lett., 1999; Shan et al, PNAS, 1999; Combeau et al, Mol. Pharmacol, 2000; Pettit et al, J. Med Chem, 2000; Pettit et al, Anticancer Drug Design, 2000; Pinney et al, Bioorg. Med. Chem. Lett., 2000; Flynn et al., Bioorg. Med. Chem. Lett., 2001; Gwaltney et al, Bioorg. Med. Chem. Lett., 2001; Lawrence et al, 2001; Nguyen-Hai et al, Bioorg. Med. Chem. Lett., 2001; Xia et al, J. Med. Chem., 2001; Tahir et al., Cancer Res., 2001; Wu-Wong et al., Cancer Res., 2001; Janik et al, Bioorg. Med. Chem. Lett., 2002; Kim et al., Bioorg Med Chem Lett., 2002; Li et al, Bioorg. Med. Chem. Lett., 2002; Nam et al, Bioorg. Med. Chem. Lett., 2002; Wang et al, J. Med. Chem. 2002; Hsieh et al, Bioorg. Med. Chem. Lett., 2003; Hadimani et al., Bioorg. Med. Chem. Lett., 2003; Mu et al, J. Med. Chem, 2003; Nam, Curr. Med. Chem., 2003; Pettit et al, J. Med. Chem., 2003; WO 02/50007, WO 02/22626, WO 02/14329, WO 01/81355, WO 01/12579, WO 01/09103, WO 01/81288, WO 01/84929, WO 00/48591, WO 00/48590, WO 00/73264, WO 00/06556, WO 00/35865, WO 00/48590, WO 99/51246, WO 99/34788, WO 99/35150, WO 99/48495, WO 92/16486, U.S. Pat. Nos. 6,433,012, 6,201,001, 6,150,407, 6,169,104, 5,731,353, 5,674,906, 5,569,786, 5,561,122, 5,430,062, 5,409,953, 5,525,632, 4,996,237 and 4,940,726 and U.S. patent application Ser. No. 10/281,528);

b) 2,3-substituted Benzo[b]thiophenes (Pinney et al, Bioorg. Med. Chem. Lett., 1999; Chen et al, J. Org. Chem., 2000; U.S. Pat. Nos. 5,886,025; 6,162,930, and 6,350,777; WO 98/39323);

c) 2,3-disubstituted Benzo[b]furans (WO 98/39323, WO 02/060872);

d) Disubstituted Indoles (Gastpar R, J. Med. Chem., 1998; Bacher et al, Cancer Res., 2001; Flynn et al, Bioorg. Med. Chem. Lett, 2001; WO 99/51224, WO 01/19794, WO 01/92224, WO 01/22954; WO 02/060872, WO 02/12228, WO 02/22576, and U.S. Pat. No. 6,232,327);

e) 2-Aroylindoles (Mahboobi et al, J. Med. Chem., 2001; Gastpar et al., J. Med. Chem., 1998; WO 01/82909)

f) 2,3-disubstituted Dihydronaphthalenes (WO 01/68654, WO 02/060872);

g) Benzamidazoles (WO 00/41669);

h) Chalcones (Lawrence et al, Anti-Cancer Drug Des, 2000; WO 02/47604)

i) Colchicine, Allocolchicine, Thiocolcichine, Halichondrin B, and Colchicine derivatives (WO 99/02166, WO 00/40529, WO 02/04434, WO 02/08213, U.S. Pat. Nos. 5,423,753. 6,423,753) in particular the N-acetyl colchinol prodrug, ZD-6126;

j) Curacin A and its derivatives (Gerwick et al, J. Org. Chem., 1994, Blokhin et al, Mol. Pharmacol., 1995; Verdier-Pinard, Arch. Biochem. Biophys., 1999; WO 02/06267);

k) Dolastatins such as Dolastatin-10, Dolastatin-15, and their analogs (Pettit et al, J. Am. Chem. Soc., 1987; Bai et al, Mol. Pharmacol, 1995; Pettit et al, Anti-Cancer Drug Des., 1998; Poncet, Curr. Pharm. Design, 1999; WO 99/35164; WO 01/40268; U.S. Pat. No. 5,985,837);

l) Epothilones such as Epothilones A, B, C, D and Desoxyepothilones A and B, Fludelone (WO 99/02514, U.S. Pat. No. 6,262,094, Nicolau et al., Nature, 1997, Pub. No. US2005/0143429);

m) Inadones (Leoni et al., J. Natl. Cancer Inst., 2000; U.S. Pat. No. 6,162,810);

n) Lavendustin A and its derivatives (Mu F et al, J. Med. Chem., 2003);

o) 2-Methoxyestradiol and its derivatives (Fotsis et al, Nature, 1994; Schumacher et al, Clin. Cancer Res., 1999; Cushman et al, J. Med. Chem., 1997; Verdier-Pinard et al, Mol. Pharmacol, 2000; Wang et al, J. Med. Chem., 2000; WO 95/04535, WO 01/30803, WO 00/26229, WO 02/42319 and U.S. Pat. Nos. 6,528,676, 6,271,220, 5,892,069, 5,661,143, and 5,504,074);

p) Monotetrahydrofurans (“COBRAs”; Uckun, Bioorg. Med. Chem. Lett., 2000; U.S. Pat. No. 6,329,420);

q) Phenylhistin and its derivatives (Kanoh et al, J. Antibiot., 1999; Kano et al, Bioorg. Med. Chem., 1999; U.S. Pat. No. 6,358,957);

r) Podophyllotoxins such as Epidophyllotoxin (Hammonds et al, J. Med. Microbiol, 1996; Coretese et al, J. Biol. Chem., 1977);

s) Rhizoxins (Nakada et al, Tetrahedron Lett., 1993; Boger et al, J. Org. Chem., 1992; Rao, et al, Tetrahedron Lett., 1992; Kobayashi et al, Pure Appl. Chem., 1992; Kobayashi et al, Indian J. Chem., 1993; Rao et al, Tetrahedron Lett., 1993);

t) 2-strylquinazolin-4(3H)-ones (“SQOs”, Jiang et al, J. Med. Chem., 1990);

u) Spongistatin and Synthetic spiroketal pyrans (“SPIKETs”; Pettit et al, J. Org. Chem., 1993; Uckun et al, Bioorgn. Med. Chem. Lett., 2000; U.S. Pat. No. 6,335,364, WO 00/00514);

v) Taxanes such as Paclitaxel (Taxol®), Docetaxel (Taxotere®), and Paclitaxel derivatives (U.S. Pat. No. 5,646,176, WIPO Publication No. WO 94/14787, Kingston, J. Nat. Prod., 1990; Schiff et al, Nature, 1979; Swindell et al, J. Cell Biol., 1981);

x) Vinca Alkaloids such as Vinblastine, Vincristine, Vindesine, Vinflunine, Vinorelbine (Navelbine®) (Owellen et al, Cancer Res., 1976; Lavielle et al, J. Med. Chem., 1991; Holwell et al, Br. J. Cancer., 2001); and

y) Welwistatin (Zhang et al, Molecular Pharmacology, 1996).

Specific examples of tubulin-binding drugs include, but are not limited to, allocolchicine, amphethinile, chelidonine, colchicide, colchicine, combrestatin A1, combretastin A4, combretastain A4 phosphate, combrestatin 3, combrestatin 4, cryptophycin, curacin A, deo-dolastatin 10, desoxyepothilone A, desoxyepothilone B, dihydroxy-pentamethoxyflananone, docetaxel, dolastatin 10, dolastatin 15, epidophyllotoxin, epothilone A, epothilone B, epothilone C, epothilone D, etoposide, fludelone, griseofulvin, halichondrin B, isocolchicine, lavendustin A, methyl-3,5-diiodo-4-(4′-methoxyphenoxy)benzoate, N-acetylcolchinol, N-acetylcolchinol-O-phosphate, N-[2-[(4-hydroxyphenyl)amino]-3-pyridyl]-4-methoxybenzenesulfonamide, nocodazole, paclitaxel, phenstatin, phenylhistin, piceid, podophyllotoxin, resveratrol, rhizoxin, sanguinarine, spongistatin 1, steganacin, taxol, teniposide, thiocolchicine, vincristine, vinblastine, welwistatin, (Z)-2-methoxy-5-[2-(3,4,5-trimethoxyphenyl)vinyl]phenylamine, (Z)-3,5,4′-trimethoxystilbene (R3), 2-aryl-1,8-naphthyridin-4(1H)-one, 2-(4′-methoxyphenyl)-3-(3′,4′,5′-trimethoxybenzoyl)-6-methoxybenzo[b]thiophene, 2-methoxy estradiol, 2-strylquinazolin-4(3H)-one, 5,6-dihydroindolo(2,1-a)isoquinoline, and 10-deacetylbaccatin III.

A “subject” is a mammal, e.g., a human, mouse, rat, guinea pig, dog, cat, horse, cow, pig, or non-human primate, such as a monkey, chimpanzee, baboon. In one embodiment, the monkey is a rhesus. In one embodiment, the subject is a human.

The phrase “pharmaceutically acceptable salt,” as used herein, is a salt formed from an acid and a base, such as an acidic or a basic salt of a molecule. The molecule in the salt can be a compound or a tubulin-binding drug. In one instance, the term “pharmaceutically acceptable salt” refers to a salt of a an acid and a basic nitrogen group of a molecule. Illustrative salts formed from an acid and a basic nitrogen group of a molecule include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, besylate, mesylate, camphor sulfonate, and pamoate (i.e., 1,1′-methylene-bis-(2-OH-3-naphthoate)) salts. The term “pharmaceutically acceptable salt” also refers to a salt of a molecule having an acidic functional group, and a pharmaceutically acceptable inorganic or organic base. Illustrative salts formed from a base and an acidic functional group of a molecule include, but are not limited to, sodium, potassium, lithium, calcium, magnesium, aluminum, zinc, ammonium; and salts with organic amines such as quaternary, tertiary, secondary, or primary organic amines, examples of which include unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, tris-(hydroxymethyl)methylamine, or 2-hydroxy-tert-butylamine, or N,N-di-lower alkyl-N-(hydroxy lower alkyl)-amines, such as N,N-dimethyl-N-(2-hydroxyethyl)amine or tri-(2-OH-ethyl)amine; N-methyl-D-glucamine; and amino acids such as arginine, lysine, and the like. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines such as tertiary, secondary, or primary organic amines, examples of which include unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, tris-(hydroxymethyl)methylamine, or 2-hydroxy-tert-butylamine, or N,N-di-lower alkyl-N-(hydroxy lower alkyl)-amines, such as N,N-dimethyl-N-(2-hydroxyethyl)amine or tri-(2-OH-ethyl)amine; N-methyl-D-glucamine; and amino acids such as arginine, lysine, and the like.

An “effective amount” when used in connection with a Panaxytriol Compound and a tubulin-binding drug is an amount of the Panaxytriol Compound or tubulin-binding drug, individually or in combination, that is effective for treating or preventing a Condition individually or in combination with another Panaxytriol Compound.

The language “in combination” includes administration within the same composition and separately. In the latter instance, the tubulin-binding drug is administered during a time when the Panaxytriol Compound exerts its prophylactic or therapeutic effect, or vice versa.

Also when administered separately, in one embodiment, the tubulin-binding drug is administered prior to administering the Panaxytriol Compound; in another embodiment, the tubulin-binding drug is administered subsequent to administering the Panaxytriol Compound; in another embodiment, the tubulin-binding drug and Panaxytriol Compound are administered concurrently.

The language “coupling agent” as used herein is a reagent that forms amide bonds, such as by coupling acids and amines In one instance, a “coupling agent” may also be referred to as a peptide coupling agent or reagent. Suitable coupling agents are well known to a person of skill in the art and are commercially available. Illustrative coupling agents include, but are not limited to, DCC, dimethylpropyl-ethylcarbodiimide (EDC), or carbonyl diimidazole (CDI). Other suitable coupling reagents will be apparent to a person of skill in the art.

The following abbreviations are used herein and have the indicated definitions: CBS is 2-methyl-oxazaborolidine, DCC is dicyclohexyl carbodiimide, DIBAL is diisobutylaluminum hydride, DMAP is N,N-dimethylaminopyridine, EDA is ethylenediamine, EtNH₂ is ethylamine, HMPA is hexamethylphosphoramide, Me is methyl, MeOH is methanol, NaH is sodium hydride, NBS is N-bromosuccinimide, TBAF is tetrabutylammonium fluoride, TBDPS is tert-butyldiphenylsilyl, TBDPSCl is tert-butyldiphenylsilyl chloride, MTPA-Cl is Mosher's acid chloride, Tf is trifluoromethanesulfonamide, THF is tetrahydrofuran, p-TsOH is para-toluenesulfonic acid, HRMS is High-Resolution Mass Spectroscopy, R_{f} is Retention Factor, and Q2D×3 means every second day for three doses.

### 5.2 Sources of Panaxytriol

Ginseng is a deciduous perennial plant that belongs to the Araliaceae family. Ginseng species include Panax ginseng, Panax quinquefolius L. (American ginseng), Panax japonicus (Japanese ginseng), Panax notoginseng (Sanchiginseng); Panax trifolius L. (Dwarf ginseng), Panax vietnamensis, and Panax pseudoginseng.

Panax ginseng can be harvested after 2 to 6 years of cultivation, and it can be classified in three ways depending on how it is processed: (a) fresh ginseng (less than 4 years old and can be consumed fresh); (b) white ginseng (4-6 years old and then dried after peeling); and (c) red ginseng (harvested when 6 years old and then steamed and dried).

Upon harvesting, ginseng can be used to make various products: for example, fresh sliced ginseng, juice, extract (tincture or boiled extract), powder, tea, tablets, and capsules.

Several components of red ginseng have been isolated and evaluated for their anticancer properties, including panaxytriol:

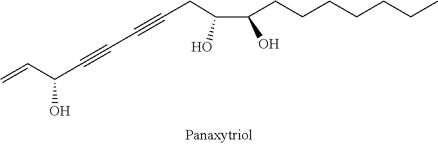

### 5.3 Methods for Making Panaxytriol Compounds

Panaxytriol can be extracted from red ginseng, for example, using ethyl acetate, and purified using chromatography on a silica gel column as described by Matsunaga et al., Chem. Pharm. Bull. 37:1279-1291 (1989).

Examples of synthetic pathways useful for making Panaxytriol Compounds are generalized in Schemes 1-5.

### 5.3.1 Panaxytriol

Compound 1 can be made by reacting n-octanal with (carbethoxymethylene)triphenylphosphorane using a Wittig reaction (see, e.g., March, Advanced Organic Chemistry: Reactions, Mechanisms, and Structure 956-963 (4th ed. 1992), followed by reduction of the ethyl ester group of the resultant product using, for example, DIBAL.

Schemes 1 and 2 set forth methodology that is useful for making panaxytriol.

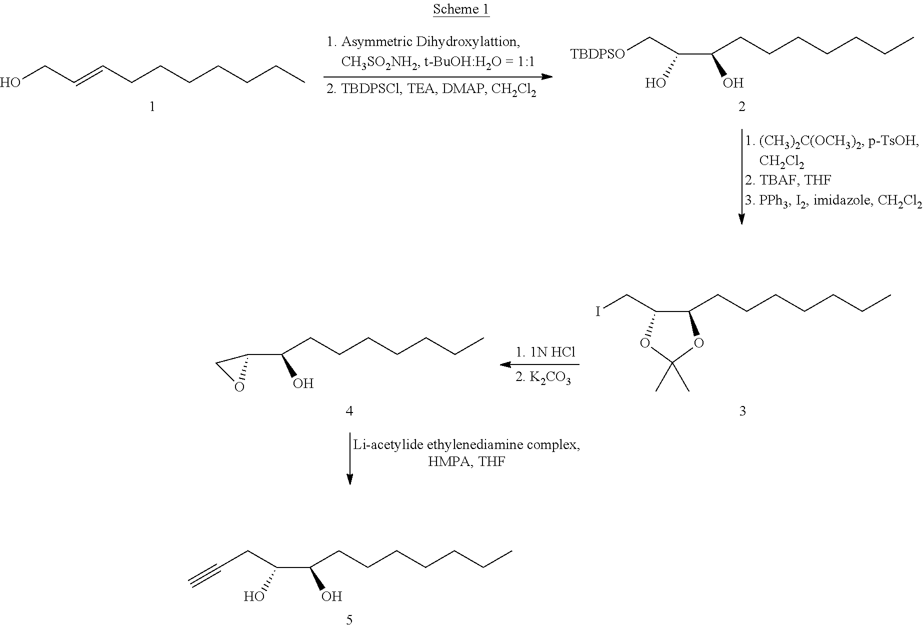

A Sharpless asymmetric dihydroxylation (Kolb et al., Chem. Rev. 94: 2483 (1994)) of compound 1 is followed by TBDPS protection of the primary alcohol to provide the diol 2. Following acetonide protection of the diol 2, the TBDPS group is removed and the resultant primary alcohol is converted to an iodide to provide the iodide 3. The iodide 3 is deprotected and treated with K₂CO₃ to provide the epoxide 4. The epoxide 4 is alkylated, for example using Li-acetylide, to provide the terminal alkyne 5.

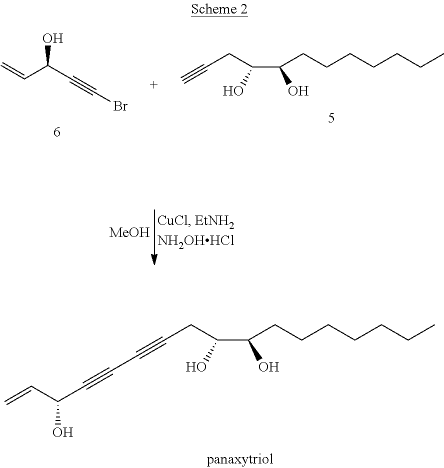

Coupling (Chodkiewicz, W. Ann. Chim. Paris, 2: 819 (1957); Randsma, L. Preparative Acetylenic Chemistry 2^{nd} Ed., Elsevier (1988); see also Siemsen et al., Angew. Chem. Int. Ed., 39: 2632 (2000)) of the alkynyl bromide 6 (prepared as described in Example 1, below) and the terminal alkyne 5, in the presence of cuprous chloride provides panaxytriol.

### 5.3.2 Compound (A)

Scheme 3 sets forth methodology useful for making Compound (A).

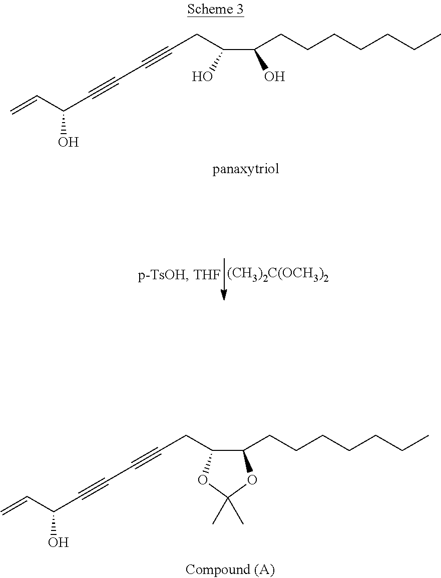

Panaxytriol can be reacted with 2,2-dimethoxypropane and a protic acid in a solvent such as THF to provide the Compound (A). Examples of a protic acid include, but are not limited to, p-Toluenesulfonic acid (p-TsOH or tosic acid), PPTS (pyridinium p-toluenesulfonate), HCl and HBr. In one embodiment, the protic acid is anhydrous. When HCl or HBr is used, it can be bubbled through the reaction mixture. In one embodiment, the amount of the protic acid is a catalytic amount. In one embodiment, the amount of the protic acid is from about 0.01 mol equivalents to about 5 mol equivalents per 1 mol of panaxytriol.

### 5.3.3 Compound (B)

Scheme 4 sets forth methodology useful for making Compound (B).

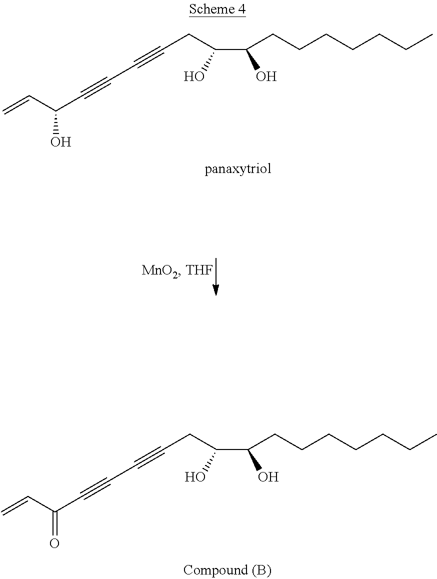

Oxidation of the allylic hydroxyl group of panaxytriol provides Compound (B). Examples of suitable oxidizing agents include, but are not limited to, MnO₂ and Dess-Martin Periodinane Reagent (see Dess and Martin (1983), J. Org. Soc., 48: 4155). In one embodiment, about 0.5 mol equivalents to about 10 mol equivalents of the oxidizing agent per 1 mol of panaxytriol is used to carry out the reaction.

### 5.3.4 Compound (C)

Scheme 5 sets forth methodology useful for making the Compound (C).

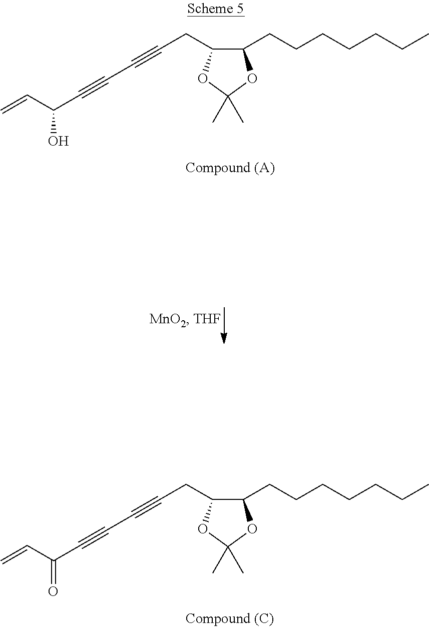

Oxidation of the allyl hydroxyl group of Compound (A) provides Compound (C). Suitable oxidizing agents include those described above for the oxidation of panaxytriol to Compound (B).

### 5.3.5 Compound (D)

Scheme 6 sets forth methodology useful for making the Compound (D).

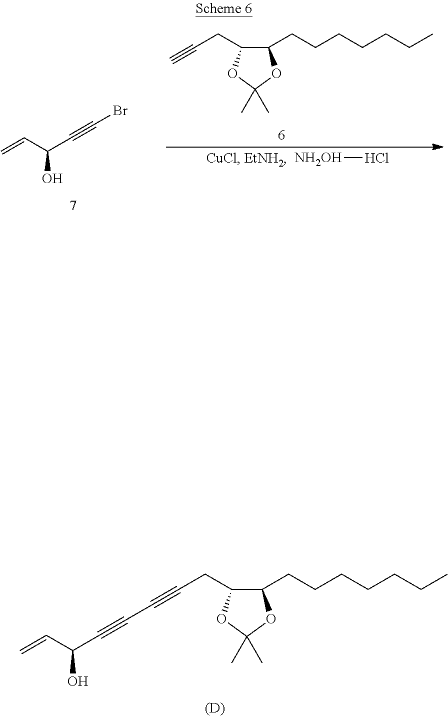

Coupling (Chodkiewicz, W. Ann. Chim. Paris, 2: 819 (1957); Randsma, L. Preparative Acetylenic Chemistry 2^{nd} Ed., Elsevier (1988); see also Siemsen et al., Angew. Chem. Int. Ed., 39: 2632 (2000)) of the alkynyl bromide 7 (prepared as described in Example 5, below) and the terminal alkyne 6, in the presence of cuprous chloride provides Compound D. The terminal alkyne 6 may be made by reacting Compound 5 with 2,2-dimethoxypropane and a protic acid neat or in a solvent such as THF to provide the Compound 6, under conditions as disclosed for making Compound (A).

### 5.3.6 Compound (E)

Scheme 7 sets forth methodology useful for making the Compound (E).

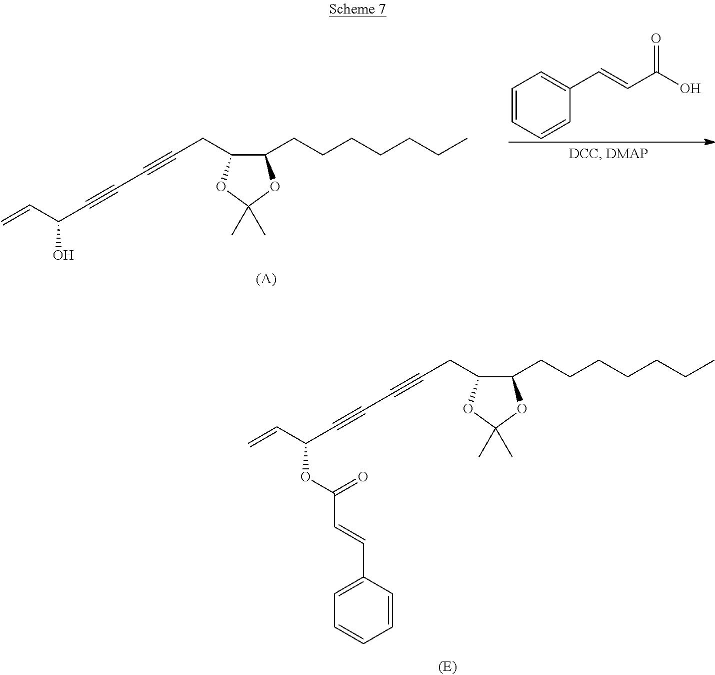

Coupling of Compound (A) with trans-cinnamic acid in the presence of a coupling agent such as DCC, optionally also in the presence of a catalyst, such as DMAP, and/or a base, such as a tertiary amine base, provides Compound (E).

### 5.37 Compound (F)

Scheme 8 sets forth methodology useful for making the Compound (F).

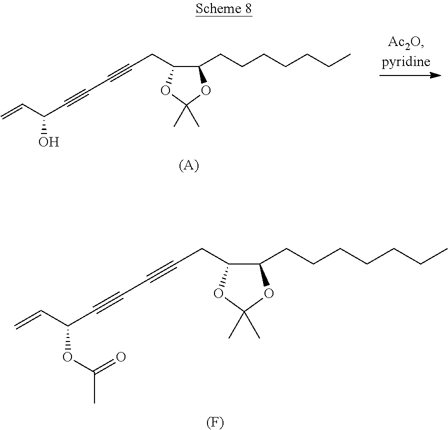

Acetylation of Compound (A) with an acyl source such as acetic anhydride in the presence of a base such as pyridine, or a tertiary amine base, optionally also in the presence of a catalyst, such as DMAP, provides Compound (F).

### 5.4 Methods for Using the Panaxytriol Compounds

In accordance with the invention, a Panaxytriol Compound and a tubulin-biding drug are administered to a subject in need of treatment or prevention of a Condition.

### 5.4.1 Methods for Treating or Preventing Cancer

A Panaxytriol Compound and a tubulin-binding drug are useful for treatment or prevention of cancer.

The invention provides methods for treating or preventing cancer, comprising administering to a subject in need of such treatment or prevention an effective amount of a Panaxytriol Compound and a tubulin-binding drug.

In one embodiment, the subject in need of treatment or prevention of cancer is considered to have a genetic risk for cancer. Examples of cancers that are associated with a genetic risk include, but are not limited to, breast cancer, colorectal cancer, uterine cancer, ovarian cancer, skin cancer and stomach cancer.

Examples of cancers that are treatable or preventable comprising administering a Panaxytriol Compound and a tubulin-binding drug include, but are not limited to, the cancers disclosed below in Table 1 and metastases thereof.

TABLE 1

```
Solid tumors, including but not limited to:
      fibrosarcoma
      myxosarcoma
      liposarcoma
      chondrosarcoma
      osteogenic sarcoma
      chordoma
      angiosarcoma
      endotheliosarcoma
      lymphangiosarcoma
      lymphangioendotheliosarcoma
      synovioma
      mesothelioma
      Ewing's tumor
      leiomyosarcoma
      rhabdomyosarcoma
      colon cancer
      colorectal cancer
      kidney cancer
      pancreatic cancer
      bone cancer
      breast cancer
      ovarian cancer
      prostate cancer
      esophageal cancer
      stomach cancer
      oral cancer
      nasal cancer
      throat cancer
      squamous cell carcinoma
      basal cell carcinoma
      adenocarcinoma
      sweat gland carcinoma
      sebaceous gland carcinoma
      papillary carcinoma
      papillary adenocarcinomas
      cystadenocarcinoma
      medullary carcinoma
      bronchogenic carcinoma
      renal cell carcinoma
      hepatoma
      bile duct carcinoma
      choriocarcinoma
      seminoma
      embryonal carcinoma
      Wilms' tumor
      cervical cancer
      uterine cancer
      testicular cancer
      small cell lung carcinoma
      bladder carcinoma
      lung cancer
      epithelial carcinoma
      head and neck cancer
      skin cancer
      melanoma
      neuroblastoma
      retinoblastoma
Leukemias:
      acute lymphoblastic leukemia (“ALL”)
      acute lymphoblastic B-cell leukemia
      acute lymphoblastic T-cell leukemia
      acute myeloblastic leukemia (“AML”)
      acute promyelocytic leukemia (“APL”)
      acute monoblastic leukemia
      acute erythroleukemic leukemia
      acute megakaryoblastic leukemia
      acute myelomonocytic leukemia
      acute nonlymphocyctic leukemia
      acute undifferentiated leukemia
      chronic myelocytic leukemia (“CML”)
      chronic lymphocytic leukemia (“CLL”)
      hairy cell leukemia
      multiple myeloma
Lymphomas:
      Hodgkin's Disease
      Multiple myeloma
      Waldenström's macroglobulinemia
      Heavy chain disease
      Polycythemia vera
CNS and brain cancers:
      glioma
      pilocytic astrocytoma
      astrocytoma
      anaplastic astrocytoma
      glioblastoma multiforme
      medulloblastoma
      craniopharyngioma
      ependymoma
      pinealoma
      hemangioblastoma
      acoustic neuroma
      oligodendroglioma
      meningioma
      vestibular schwannoma
      adenoma
      metastatic brain tumor
      meningioma
      spinal tumor
      medulloblastoma
```

In one embodiment the cancer is lung cancer, breast cancer, colorectal cancer, prostate cancer, a leukemia, a lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment the cancer is metastatic cancer.

In another embodiment, the cancer is an indolent cancer, such as prostate cancer, breast cancer, lung cancer or a lymphoma.

In still another embodiment, the subject has previously undergone or is presently undergoing treatment for cancer. Such previous treatments include, but are not limited to, prior chemotherapy, radiation therapy, surgery or immunotherapy, such as cancer vaccines.

A Panaxytriol Compound and a tubulin-binding drug are also useful for the treatment or prevention of a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., El-Serag, J Clin Gastroenterol (2002) 35(5 Suppl 2):572-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11):1574-9); and Human Immune deficiency Virus (HIV) infection, which can lead to cancer as a consequence of immunodeficiency (see, e.g., Dal Maso et al., Lancet Oncol (2003) 4(2):110-9).

A Panaxytriol Compound and a tubulin-binding drug can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic/therapeutic administration of a Panaxytriol Compound and a tubulin-binding drug. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also Id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, is treatable or preventable according to the present methods.

In another embodiment, fibrocystic disease (cystic hyperplasia, mammary dysplasia, particularly adenosis (benign epithelial hyperplasia)) is treatable or preventable according to the present methods.

In other embodiments, a subject that exhibits one or more of the following predisposing factors for malignancy can be administered with an effective amount of a Panaxytriol Compound and a tubulin-binding drug: a chromosomal translocation associated with a malignancy (e.g., the Philadelphia chromosome for chronic myelogenous leukemia, t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome); and exposure to carcinogens (e.g., smoking, second-hand smoke exposure, and inhalation of or contacting with certain chemicals).

Administration of an effective amount of a Panaxytriol Compound and a tubulin-binding drug is also useful for the maintenance therapy of cancer. Maintenance therapy can help keep cancer under control and help keep a subject disease free for an extended period of time.

In one embodiment, maintenance therapy is administered to a subject that is in remission.

Administration of an effective amount of a Panaxytriol Compound and a tubulin-binding drug is also useful for treating a micrometastasis. In one embodiment, the subject is treated for a micrometastasis after the subject achieves remission after being treated with chemotherapy, radiation therapy, surgery, or a combination thereof.

In addition, administration of an effective amount of a Panaxytriol Compound and a tubulin-binding drug is useful for preventing a micrometastasis. Without being bound by theory, it is believed that a micrometastasis is therapeutically suppressible by a variety of mechanisms including direct tumor cell kill, cytotoxic disruption of paracrine growth signals from normal tissues, and targeted inhibition of prometastatic pathways.

In one embodiment, a Panaxytriol Compound and a tubulin-binding drug are administered at doses commonly employed when such agents are used as monotherapy for the treatment of cancer.

In another embodiment, a Panaxytriol Compound and a tubulin-binding drug act synergistically and are administered at doses that are less than the doses commonly employed when such agents are used as monotherapy for the treatment of cancer.

The dosage of a Panaxytriol Compound, and a tubulin-binding drug administered as well as the dosing schedule can depend on various parameters, including, but not limited to, the cancer being treated, the patient's general health, and the administering physician's discretion.

A Panaxytriol Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a tubulin-binding drug to a subject in need thereof. In various embodiments, a Panaxytriol Compound, and a tubulin-binding drug are administered 5 seconds apart, 15 seconds apart, 30 seconds apart, 1 minute apart, 5 minutes apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one embodiment, a Panaxytriol Compound and a tubulin-binding drug are administered within 3 hours of each other. In another embodiment, a Panaxytriol Compound and a tubulin-binding drug are administered 1 minute to 24 hours apart.

In one embodiment, a Panaxytriol Compound and a tubulin-binding drug are present in the same composition. In one embodiment, this composition is useful for oral administration. In another embodiment, this composition is useful for intravenous administration.

Cancers that can be treated or prevented by administering a Panaxytriol Compound and a tubulin-binding drug include, but are not limited to, the list of cancers set forth in Table 1.

The Panaxytriol Compound and the tubulin-binding drug can act additively or synergistically. A synergistic combination of a Panaxytriol Compound and a tubulin-binding drug might allow the use of lower dosages of one or both of these agents, and/or less frequent dosages of one or both of the Panaxytriol Compound and a tubulin-binding drug, and/or less frequent administration of the agents could reduce any toxicity associated with the administration of the agents to a subject; without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, a Panaxytriol Compound and a tubulin-binding drug act synergistically when administered in doses typically employed when such agents are sued as monotherapy for the treatment of cancer. In another embodiment, a Panaxytriol Compound and a tubulin-binding drug act synergistically when administered in doses that are less than doses typically employed when such agents are used as monotherapy for the treatment of cancer.

In some embodiments, administration of a Panaxytriol Compound reduces the effective amount of a tubulin-binding drug by 2-fold, 5-fold, 10-fold, 20-fold, 50-fold, 100-fold, or 1000-fold. Reduction of the effective amount of tubulin-binding drug can result in reduction of adverse side-effects associated with administration of the tubulin-binding drug.

In one embodiment, the tubulin-binding drug is administered orally.

In another embodiment, the tubulin-binding drug is administered intravenously.

### 5.4.2 Combination Chemotherapy

In one embodiment, the methods for treating or preventing cancer further comprise administering an effective amount of another anticancer agent.

In one embodiment, the other anticancer agent useful in the methods and compositions of the present invention includes, but is not limited to, a drug listed in Table 2 or a pharmaceutically acceptable salt thereof.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum complexes: | Cisplatin |
| | Carboplatin |
| | Aroplatin |
| | Oxaliplatin |
| DNA Topoisomerase Inhibitors | |
| Epipodophyllins: | Topotecan |
| | Irinotecan |
| | 9-aminocamptothecin |
| | Camptothecin |
| | Crisnatol |
| Mitomycins: | Mitomycin C |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase Inhibitors: | Hydroxyurea |
| | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine |
| | Cytosine arabinoside |
| | Fludarabine |
| | Gemcitabine |
| | Capecitabine |
| Purine analogs: | Mercaptopurine |
| | Thioguanine |
| | O-6-benzylguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goserelin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interleukins |
| | Kringle 5 (plasminogen fragment) |
| | Marimastat |
| | Metalloproteinase inhibitors |
| | 2-Methoxyestra diol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors (FTI) |
| | Bisphosphonates |
| Antimitotic agents: | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

In another embodiment, additional other anticancer agents useful in the methods and compositions of the present invention include, but are not limited to, the following compounds or a pharmaceutically acceptable salt thereof: abiraterone, acivicin, aclarubicin, acodazole, acronine, acylfulvene, adecypenol, adozelesin, aldesleukin, an ALL-TK antagonist, altretamine, ambamustine, ambomycin, ametantrone, amidox, amifostine, aminoglutethimide, aminolevulinic acid, amrubicin, amsacrine, anagrelide, anastrozole, andrographolide, an angiogenesis inhibitor, antarelix, anthramycin, an apoptosis gene modulator, apurinic acid, ara-CDP-DL-PTBA, arginine deaminase, L-asparaginase, asperlin, asulacrine, atamestane, atrimustine, axinastatin 1, axinastatin 2, axinastatin 3, azacitidine, azasetron, azatoxin, azetepa, azatyrosine, azotomycin, batimastat, benzodepa, bisantrene, bisnafide, bizelesin, brequinar, bropirimine, balanol, a BCR/ABL antagonist, beta-alethine, betaclamycin B, betulinic acid, bisaziridinylspermine, bisnafide, bistratene A, bizelesin, calcipotriol, calphostin C, calusterone, canarypox IL-2, carubicin, carboxyamidotriazole, CaRest M3, CARN 700, carzelesin, castanospermine, cecropin B, cetrorelix, chloroquinoxaline, cicaprost, cirolemycin, cladribine, clotrimazole, collismycin A, collismycin B, conagenin, crambescidin 816, crisnatol, cryptophycin 8, cryptophycin A derivatives, cyclopentanthraquinones, cycloplatam, cypemycin, cytostatin, dacliximab, decitabine, dehydrodidemnin B, deslorelin, dexifosfamide, dexormaplatin, dexrazoxane, dexdiaziquone, didemnin B, didox, diethylnorspermine, dihydro-5-acytidine, dihydrotaxol, dioxamycin, diphenyl spiromustine, docosanol, dolasetron, droloxifene, dronabinol, duazomycin, duocarmycin SA, ecomustine, edatrexate, eflornithine, elsamitrucin, enloplatin, enpromate, epipropidine, erbulozole, esorubicin, estramustine, estramustine, an estrogen antagonist, etanidazole, etoprine, exemestane, fadrozole, fazarabine, fenretinide, finasteride, flavopiridol, flezelastine, fluasterone, fluorodaunorunicin, floxuridine, flurocitabine, forfenimex, formestane, fostriecin, fotemustine, gadolinium texaphyrin, galocitabine, ganirelix, a gelatinase inhibitor, a glutathione inhibitor, hepsulfam, herbimycin A, heregulin, hexamethylene bisacetamide, hypericin, ibandronic acid, idoxifene, idramantone, ilmofosine, ilomastat, imatinib mesylate, imidazoacridones, imiquimod, an IGF-1 inhibitor, iobenguane, iodoipomeanol, iproplatin, irsogladine, isobengazole, isohomohalicondrin B, itasetron, jasplakinolide, leucovorin, levamisole, leuprorelin, liarozole, lissoclinamide 7, lobaplatin, lombricine, lometrexol, lonidamine, losoxantrone, lovastatin, loxoribine, lurtotecan, lutetium texaphyrin, lysofylline, mannostatin A, masoprocol, maspin, a matrix metalloproteinase inhibitor, mechlorethamine, megestrol acetate melphalan, metoclopramide, mifepristone, miltefosine, mirimostim, mitoguazone, mitolactol, mitonafide, mofarotene, molgramostim, mopidamol, a multiple drug resistance gene inhibitor, myriaporone, N-acetyldinaline, nafarelin, nagrestip, napavin, naphterpin, nartograstim, nedaplatin, nemorubicin, neridronic acid, nilutamide, nisamycin, a nitrogen mustard, a nitric oxide modulator, a nitrosourea, nitrullyn, octreotide, okicenone, onapristone, oracin, ormaplatin, osaterone, oxaunomycin, palauamine, palmitoylpamidronic acid, panaxytriol, panomifene, parabactin, pazelliptine, pegaspargase, peldesine, peliomycin, pentamustine, pentosan, pentostatin, pentrozole, peplomycin, perfosfamide, perflubron, perfosfamide, phenazinomycin, a phosphatase inhibitor, picibanil, pilocarpine, pipobroman, piposulfan, piritrexim, placetin A, placetin B, plicamycin, porfiromycin, plomestane, porfimer sodium, porfiromycin, prednimustine, prednisone, prostaglandin J2, microalgal, puromycin, pyrazoloacridine, pyrazofurin, a raf antagonist, raltitrexed, ramosetron, a ras farnesyl protein transferase inhibitor, a ras-GAP inhibitor, retelliptine demethylated, RII retinamide, riboprine, rogletimide, rohitukine, romurtide, roquinimex, rubiginone B1, ruboxyl, safingol, saintopin, SarCNU, sarcophytol A, sargramostim, semustine, a signal transduction modulator, simtrazene, sizofiran, sobuzoxane, solverol, sonermin, sparfosic acid, sparfosate, sparsomycin, spicamycin D, spiromustine, spiroplatin, splenopentin, a stem-cell division inhibitor, stipiamide, streptonigrin, a stromelysin inhibitor, sulfinosine, suradista, suramin, swainsonine, talisomycin, tallimustine, tauromustine, tazarotene, tecogalan, tegafur, tellurapyrylium, a telomerase inhibitor, teloxantrone, temoporfin, teroxirone, testolactone, tetrachlorodecaoxide, tetrazomine, thaliblastine, thiamiprine, thiocoraline, thrombopoietin, thymalfasin, thymotrinan, tirapazamine, titanocene, topsentin, toremifene, trestolone, tretinoin, triacetyluridine, triciribine, trimetrexate, triptorelin, tropisetron, tubulozole, turosteride, a tyrosine kinase inhibitor, ubenimex, uracil mustard, uredepa, vapreotide, variolin B, velaresol, veramine, verteporfin, vinxaltine, vinepidine, vinglycinate, vinleurosine, vinrosidine, vinzolidine, vitaxin, vorozole, zanoterone, zeniplatin, zilascorb, zinostatin, and zorubicin.

### 5.4.3 Multi-Therapy for Cancer

A Panaxytriol Compound and a tubulin-binding drug can be administered to a subject that has undergone or is currently undergoing one or more additional anticancer therapies including, but not limited to, surgery, radiation therapy, or immunotherapy, such as a cancer vaccine.

In one embodiment, the invention provides methods for treating or preventing cancer comprising administering to a subject in need thereof (a) an amount of a Panaxytriol Compound and a tubulin-binding drug effective to treat or prevent cancer; and (b) another anticancer therapy including, but not limited to, surgery, radiation therapy, or immunotherapy, such as a cancer vaccine.

A Panaxytriol Compound or a tubulin-binding drug can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer therapy to a subject in need thereof. In various embodiments, (a) a Panaxytriol Compound and a tubulin-binding drug, and (b) another anticancer therapy are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, no more than 48 hours apart, no more than one week apart, no more than two weeks apart, no more than three weeks apart, no more than one month apart, no more than two months apart, no more than three months apart or no more than six months apart. In one embodiment, (a) a Panaxytriol Compound and a tubulin-binding drug, and (b) another anticancer therapy are administered within 3 hours of each other. In another embodiment, (a) a Panaxytriol Compound and a tubulin-binding drug, and (b) another anticancer therapy are administered 1 minute to 24 hours apart.

In one embodiment, the other anticancer therapy is radiation therapy.

In another embodiment, the other anticancer therapy is surgery.

In still another embodiment, the other anticancer therapy is immunotherapy.

In another embodiment, the other anticancer therapy is hormonal therapy.

In a specific embodiment, the present methods for treating or preventing cancer comprise administering a Panaxytriol Compound, a tubulin-binding drug and radiation therapy. The radiation therapy can be administered concurrently with, prior to, or subsequent to the Panaxytriol Compound or tubulin-binding drug; in one embodiment, at least an hour, five hours, 12 hours, a day, a week, a month; in another embodiment, several months (e.g., up to three months), prior or subsequent to administration of the Panaxytriol Compound or a tubulin-binding drug.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for a deep tumor, and electron beam and orthovoltage X-ray radiation can be used for skin cancer. A gamma-ray emitting radioisotope, such as a radioactive isotope of radium, cobalt and other element, can also be administered.

Additionally, in one embodiment the invention provides methods for treating cancer comprising administering a Panaxytriol Compound and a tubulin-binding drug as an alternative to chemotherapy or radiation therapy where the chemotherapy or the radiation therapy results in negative side effects, in the subject being treated. The subject being treated can, optionally, be treated with another anticancer therapy such as surgery, radiation therapy, or immunotherapy.

The Panaxytriol Compound and a tubulin-binding drug can also be used in vitro or ex vivo, such as for the treatment of certain cancers, including, but not limited to leukemias and lymphomas, wherein such treatment involves an autologous stem cell transplant. This can involve a process in which the subject's autologous hematopoietic stem cells are harvested and purged of all cancer cells by administration of a Panaxytriol Compound and a tubulin-binding drug and/or radiation, and the resultant stem cells are infused back into the subject. Supportive care can be subsequently provided while bone marrow function is restored and the subject recovers.

### 5.4.4 Methods for Treating or Preventing a Neurotrophic Disorder

Administration of an effective amount of a Panaxytriol Compound and a tubulin-binding drug can be used to treat or prevent a neurotrophic disorder.

Accordingly, the invention provides methods for treating or preventing a neurotrophic disorder, comprising administering to a subject in need of such treatment or prevention an effective amount of a Panaxytriol Compound and a tubulin-binding drug.

Examples of neurotrophic disorders that are treatable or preventable using a Panaxytriol Compound and a tubulin-binding drug include, but are not limited to, neutrotrophic atrophy; neurotrophic keratitis; a disease associated with cognitive dysfunction, such as dementia or Alzheimer's disease; a neurodegenerative disease, such as amyotrophic lateral sclerosis or stroke; a pain disorder, such as neuropathic pain or cancer pain; a psychotic disorder such as schizophrenia; a movement disorder, such as Parkinson's disease; or a seizure disorder, such as temporal lobe epilepsy.

In one embodiment, the neurotrophic disorder is a disease associated with cognitive dysfunction.

In another embodiment, the neurotrophic disorder is a neurodegenerative disease.

In yet another embodiment, the neurotrophic disorder is a pain disorder.

In another embodiment, the neurotrophic disorder is a psychotic disorder.

In a further embodiment, the neurotrophic disorder is a movement disorder.

In another embodiment, the neurotrophic disorder is a seizure disorder.

### 5.4.5 Therapeutic/Prophylactic Administration

In one embodiment, the invention provides compositions useful for treating or preventing a Condition. The compositions are suitable for internal or external use and comprise a physiologically acceptable carrier or vehicle and an effective amount of a Panaxytriol Compound and a tubulin-binding drug.

A Panaxytriol Compound and a tubulin-binding drug can be administered in amounts that are effective to treat or prevent a Condition in a subject.

Administration of a Panaxytriol Compound and a tubulin-binding drug can be accomplished via any mode of administration for therapeutic agents. These modes include systemic or local administration such as oral, nasal, parenteral, transdermal, subcutaneous, vaginal, buccal, rectal or topical administration modes.

In one embodiment, a Panaxytriol Compound and a tubulin-binding drug are administered orally.

In embodiment, when the Panaxytriol Compound is panaxytriol, it is administered by oral administration of a root of a Panax genus, or an extract thereof, and the tubulin-binding drug is administered separately. Oral administration of a root of a Panax genus can comprise ingesting the root of a Panax genus, or an extract thereof. In this embodiment, the tubulin-binding drug is administered separately, either before, after, or concurrently with ingestion of the root of a Panax genus or an extract thereof. In this embodiment, the mode of administration of the tubulin-binding drug can be any mode suitable for administration of the tubulin-binfing drug.

Depending on the intended mode of administration, compositions comprising an effective amount of a Panaxytriol Compound and a tubulin-binding drug can be in solid, semi-solid or liquid dosage form, such as, for example, injectables, tablets, suppositories, pills, time-release capsules, elixirs, tinctures, emulsions, syrups, powders, liquids, suspensions, or the like, in one embodiment in unit dosages and consistent with conventional pharmaceutical practices. Likewise, the compositions can also be administered in intravenous (both bolus and infusion), intraperitoneal, subcutaneous or intramuscular form, all using other forms known to those skilled in the art.

Illustrative pharmaceutical compositions include tablets and gelatin capsules. Illustrative carriers or vehicles include a) a diluent, e.g., lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; and/or e) absorbent, colorant, flavorant and sweetener.

Liquid, particularly injectable, compositions can, for example, be prepared by dissolution or dispersion. For example, a Panaxytriol Compound and a tubulin-binding drug are admixed with a pharmaceutically acceptable solvent such as, for example, water, saline, aqueous dextrose, glycerol, ethanol, and the like, to thereby form an injectable isotonic solution or suspension.

A Panaxytriol Compound and a tubulin-binding drug can be also formulated as a suppository that can be prepared from fatty emulsions or suspensions, using polyalkylene glycols such as propylene glycol, as the carrier.

A Panaxytriol Compound and a tubulin-binding drug can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

A Panaxytriol Compound and a tubulin-binding drug can also be delivered by the use of monoclonal antibodies as individual carriers to which the Panaxytriol Compound molecules and and tubulin-binding drugs are coupled. The Panaxytriol Compounds and tubulin-binding drugs can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Panaxytriol Compounds and tubulin-binding drugs can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Parental injectable administration can be used for subcutaneous, intramuscular or intravenous injections and infusions. Injectables can be prepared in conventional forms, either as liquid solutions or suspensions or solid forms suitable for dissolving in liquid prior to injection.

In one embodiment, the Panaxytriol Compound and a tubulin-binding drug are administered intravenously.

One embodiment, for parenteral administration employs the implantation of a slow-release or sustained-released system, according to U.S. Pat. No. 3,710,795, incorporated herein by reference.

The compositions can be sterilized or can contain non-toxic amounts of adjuvants, such as preserving, stabilizing, wetting or emulsifying agents, solution promoters, salts for regulating the osmotic pressure pH buffering agents, and other substances, including, but not limited to, sodium acetate or triethanolamine oleate. In addition, the compositions can also contain other therapeutically useful substances.

Compositions can be prepared according to conventional mixing, granulating or coating methods, respectively, and the present pharmaceutical compositions can contain from about 0.1% to about 99%, preferably from about 1% to about 70% of the Panaxytriol Compound and a tubulin-binding drug by weight or volume.

The dosage regimen utilizing the Panaxytriol Compound and a tubulin-binding drug can be selected in accordance with a variety of factors including type, species, age, weight, sex and medical condition of the subject; the severity of the Condition; the route of administration; the renal or hepatic function of the subject; and the particular Panaxytriol Compound or tubulin-binding drug employed. A person skilled in the art can readily determine or prescribe the effective amount of the Panaxytriol Compound or tubulin-binding drug useful for treating or preventing a Condition.

Effective dosage amounts of a Panaxytriol Compound, when administered to a subject, range from about 0.05 to about 1000 mg of the Panaxytriol Compound per day. Compositions for in vivo or in vitro use can contain about 0.5, 1.0, 2.5, 5.0, 10.0, 15.0, 25.0, 50.0, 100.0, 250.0, 500.0 or 1000.0 mg of Panaxytriol Compound. In one embodiment, the compositions are in the form of a tablet that can be scored. Effective plasma levels of the Panaxytriol Compounds can range from about 0.002 mg to about 50 mg per kg of body weight per day. The amount of a Panaxytriol Compound that, in combination with a tubulin-binding drug, is effective for the treatment or prevention of a Condition can be determined using clinical techniques that are known to those of skill in the art. In addition, in vitro and in vivo assays can optionally be employed to help identify optimal dosage ranges. The precise dose to be employed can also depend on the route of administration, and the seriousness of the Condition and can be decided according to the judgment of the practitioner and each subject's circumstances in view of, e.g., published clinical studies. Suitable effective dosage amounts, however, can range from about 10 micrograms to about 5 grams about every 4 hours, in one embodiment about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Panaxytriol Compound is administered, the effective dosage amounts correspond to the total amount administered.

Effective dosage amounts of a tubulin-binding drug, when administered to a subject, range from about 0.05 to about 1000 mg of the tubulin-binding drug per day. Compositions for in vivo or in vitro use can contain about 0.5, 1.0, 2.5, 5.0, 10.0, 15.0, 25.0, 50.0, 100.0, 250.0, 500.0 or 1000.0 mg of the tubulin-binding drug. In one embodiment, the compositions are in the form of a tablet that can be scored. Effective plasma levels of the the tubulin-binding drug can range from about 0.002 mg to about 50 mg per kg of body weight per day. The amount of a tubulin-binding drug that, in combination with a Panaxytriol Compound, is effective for the treatment or prevention of a Condition can be determined using clinical techniques that are known to those of skill in the art. In addition, in vitro and in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, can range from about 10 micrograms to about 5 grams about every 4 hours, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one tubulin-binding drug is administered, the effective dosage amounts correspond to the total amount administered.

A Panaxytriol Compound and a tubulin-binding drug can be administered in a single daily dose, or the total daily dosage can be administered in divided doses of two, three or four times daily. Furthermore, a Panaxytriol Compound and a tubulin-binding drug can be administered in intranasal form via topical use of suitable intranasal vehicles, or via transdermal routes, using those forms of transdermal skin patches known to those of skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of a Panaxytriol Compound and a tubulin-binding drug ranges from about 0.1% to about 15%, weight/weight or weight/volume.

In one embodiment, the compositions comprise a total amount of a Panaxytriol Compound and a tubulin-binding drug that is effective to treat or prevent a Condition. In another embodiment, the amount of Panaxytriol Compound and the tubulin-binding drug is at least about 0.01% of the combined combination chemotherapy agents by weight of the composition. When intended for oral administration, this amount can be varied from about 0.1% to about 80% by weight of the composition. Some oral compositions can comprise from about 4% to about 50% of a Panaxytriol Compound and a tubulin-binding drug. Other compositions of the present invention are prepared so that a parenteral dosage unit contains from about 0.01% to about 2% by weight of the composition.

The Panaxytriol Compounds and tubulin-binding drugs can be assayed in vitro or in vivo for the desired therapeutic or prophylactic activity prior to use in humans Animal model systems can be used to demonstrate safety and efficacy.

The present methods for treating or preventing a Condition in a subject in need thereof can further comprise administering another prophylactic or therapeutic agent to the subject being administered a Panaxytriol Compound and a tubulin-binding drug. In one embodiment the other prophylactic or therapeutic agent is administered in an effective amount. The other prophylactic or therapeutic agent includes, but is not limited to, an antiemetic agent, a hematopoietic colony stimulating factor, an anxiolytic agent, and an analgesic agent.

In a further embodiment, the Panaxytriol Compound and a tubulin-binding drug can be administered prior to, concurrently with, or after the other prophylactic or therapeutic agent, or on the same day, or within 1 hour, 2 hours, 12 hours, 24 hours, 48 hours or 72 hours of each other.

Effective amounts of the prophylactic or therapeutic agents are known to those skilled in the art. However, it is well within the skilled artisan's purview to determine the other prophylactic or therapeutic agent's optimal effective amount range. In one embodiment of the invention, the effective amount of the Panaxytriol Compound is less than its effective amount would be where the tubulin-binding drug is not administered. In this case, without being bound by theory, it is believed that the Panaxytriol Compound and the tubulin-binding drug act synergistically to treat or prevent a Condition.

In one embodiment, the other prophylactic or therapeutic agent is an antiemetic agent. Antiemetic agents useful in the methods of the present invention include include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine and tropisetron.

In one embodiment, the other prophylactic or therapeutic agent is a hematopoietic colony stimulating factor. Hematopoietic colony stimulating factors useful in the methods of the present invention include, but are not limited to, filgrastim, sargramostim, molgramostim and epoietin alfa.

In one embodiment, the other prophylactic or therapeutic agent is an opioid analgesic agent. Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

In one embodiment, the other prophylactic or therapeutic agent is a non-opioid analgesic agent. Non-opioid analgesic agents useful in the methods of the present invention include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In one embodiment, the other prophylactic or therapeutic agent is an anxiolytic agent. Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

The compositions of the invention can be sold or used as prescription products, or alternatively as over-the-counter products. In one embodiment, the compositions of the invention can be sold or used as nutraceutical products.

### 5.5 Kits

The invention encompasses kits that can simplify the administration of a Panaxytriol Compound and a unit dosage form of a tubulin-binding drug to a subject.

A typical kit of the invention comprises a unit dosage form of a Panaxytriol Compound and a unit dosage form of a tubulin-binding drug. In one embodiment the unit dosage form is a container, which can be sterile, containing an effective amount of a Panaxytriol Compound and a tubulin-binding drug and a physiologically acceptable carrier or vehicle. The kit can further comprise a label or printed instructions instructing the use of the Panaxytriol Compound and a tubulin-binding drug to treat or prevent a Condition. The kit can also further comprise a unit dosage form of another prophylactic or therapeutic agent, for example, a container containing an effective amount of the other prophylactic or therapeutic agent. In one embodiment the kit comprises a container containing an effective amount of a Panaxytriol Compound and a tubulin-binding drug and an effective amount of another prophylactic or therapeutic agent. Examples of other therapeutic agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag.

The invention is further described in the following examples, which do not limit the scope of the invention described in the claims. The following examples illustrate the synthesis of Panaxytriol Compounds and demonstrate their usefulness in combination with a tubulin-binding drug for treating or preventing a Condition.

### 6. EXAMPLES

General Synthetic Methods

All commercial materials were used without further purification unless otherwise noted. THF, diethyl ether and methylene chloride used as reaction solvents were obtained from a dry system (alumina) and used without further drying. Hexamethylphosphoramide was freshly distilled over calcium hydride under vacuum. All reactions were performed under a positive pressure of argon atmosphere in flame-dried vessels. ¹H spectra were obtained on a DRX-400 MHz Bruker instrument and are reported in parts per million (δ) from residual non deuterated solvent as an internal reference. ¹³C NMR spectra were recorded on AMX-75 MHz Bruker instruments and are reported in parts per million (δ) from residual non deuterated solvent as an internal reference. Infrared (IR) spectra were taken as a thin film on a Perkin Elmer FT-IR Spectrometer Paragon 1000. Optical rotations were recorded on a Jasco DIP-1000 polarimeter using a 1 dm cell at the reported temperature and concentrations. High resolution mass spectra were recorded on a JEOL-DX-303 HF mass spectrometer. Analytical thin layer chromatography was performed on E. Merck silica gel 60 F254 plates (0.25 mm) Liquid column chromatography was performed using forced flow of a mixture of solvents on E. Merk silica gel 60 (40-63 mm) Purification by preparative Thin Layer Chromatography (TLC) was performed using silica gel GF plates (1000 microns). When required, the stereochemistry was established by suitable one-dimensional or multi-dimensional NMR studies.

### 6.1 Example 1

### Synthesis of Panaxytriol

A. Synthesis of Alkynyl Bromide 6

(R)-Me-CBS reagent (1.0 M Toluene, 2.14 mL, 2.14 mmol) was transferred into a freshly flame-dried flask, and the toluene was removed in vacuo over 1 day. The CBS reagent was diluted with a THF solution of 5-trimethylsilyl-1-penten-4-yn-3-one (163 mg, 1.07 mmol), and the resultant solution was cooled to −30° C. At −30 ° C., BH₃-Me₂S (0.589 mL, 1.18 mmol) was slowly added over 10 min. After addition of BH₃-Me₂S, TLC analyses indicated that the reaction was complete. Methanol was slowly added, and reaction mixture was slowly warmed to room temperature. The reaction mixture was diluted with diethyl ether, and the resultant organic phase was washed with 2:1 (v:v) NaOH/saturated NaHCO₃ solution until the aqueous phase was clear, and then washed with brine. After being dried over MgSO₄, the organic phase was removed, diluted with diethyl ether, and to this was added a solution of 0.5 M HCl in methanol (4.5 mL, 2.14 mmol). Precipitates were removed by filtration. The crude product in the solvent mixture was purified by flash column chromatography (hexane/ether 5:1) to provide (3R)-5-trimethylsilyl-1-penten-4yn-3-ol (0.163 g, 100% yield) as a colorless oil: R_{f}: 0.4 (hexane/dichloromethane 2:1); [α]_{D}^{20.0°}: −24.1 (c=1, CHCl₃); ¹H NMR (400 MHz, CDCl₃): δ 5.95 (ddd, 1H, J=17.0, 10.1, 5.29 Hz), 5.46 (d, 1H, J=7.0 Hz), 5.21 (d, 1H, J=10.1 Hz), 4.86 (d, 1H, J=3.87 Hz), 2.17 (br s, 1H), 0.16 (s, 9H); ¹³C NMR (75 MHz, CDCl₃) δ 137.0, 116.8, 104.9, 91.3, 63.9, 0.2; IR (neat) v: 3368.7, 2961.3, 2927.0, 2855.3, 2174.4, 1250.9, 843.7 cm⁻¹; HRMS: calculated 154.28, found 154.0817 for [M]⁺.

Following protocols in Sullivan et al. (1973), J. Org. Chem. 38:2143; and Ohtani et al. (1991), J. Am. Chem. Soc. 113: 4092 a Mosher ester derived from (3R)-5-trimethylsilyl-1-penten-4yn-3-ol was prepared using (R)-MTPA-Cl. The ¹H NMR signals (δ 6.091, 5.868) of the Mosher ester of (3R)-5-trimethylsilyl-1-penten-4yn-3-ol appeared at higher fields than those (δ 6.119 and 5.958) of the S-isomer ((3S)-5-trimethylsilyl-1-penten-4yn-3-ol). The resultant (3R)-5-trimethylsilyl-1-penten-4yn-3-ol (204 mg, 1.32 mmol) was dissolved in acetone. NBS (353 mg, 1.98 mmol) and silver nitrate (45 mg, 0.26 mmol) were added to this solution. The reaction mixture was stirred at room temperature for 1 hr. The mixture was cooled to 0° C., mixed with cold water, and extracted with diethyl ether. The extract was washed with water and brine, dried over MgSO₄, and concentrated under reduced pressure. The residue was purified by flash column chromatography (hexane/ether 4:1) to provide compound 6 (212 mg, 100%) as a colorless oil: R_{f}: 0.49 (hexane/ether 2:1); [α]_{D}^{20.4°}: −31.61 (c=1, CHCl₃); ¹H NMR (400 MHz, CDCl₃): δ 5.94 (m, 1H), 5.47 (d, 1H, J=17.0 Hz), 5.24 (d, 1H, J=10.1 Hz), 4.88 (d, 1H, J=5.34 Hz), 2.44 (br s, 1H); ¹³C NMR (75 MHz, CDCl₃): δ 136.7, 117.4, 79.3, 64.3, 47.2; IR (neat): ν 3361.2, 2918.7, 2852.9, 2356.6 cm⁻¹; HRMS: calculated 161.00, found 161.0334 for [M]⁺.

B. Synthesis of Terminal Alkyne 5

To a solution of lithium acetylide-EDA complex (0.330 g, 3.58 mmol) in THF and HMPA (0.2 mL) was added the epoxide 4 (0.206 g, 1.19 mmol) at 0° C. The reaction mixture was stirred at that temperature overnight, quenched with saturated ammonium chloride, extracted with ethyl acetate, washed with brine, dried over MgSO₄, and concentrated under reduced pressure. The residue was purified using flash column chromatography (hexane/ethyl acetate 4:1) to provide the terminal alkyne 5 (0.189 g, 80%) as a yellow oil: R_{f} 0.24 (hexane/ethyl acetate 3:1); [α]_{D}^{19.7°}+0.1131 (c=1, CHCl₃); ¹H NMR (400 MHz, CDCl₃): δ 3.61 (m, 2H), 2.47 (m, 2H), 2.31 (br s, 2H), 2.06 (s, 1H), 1.50-1.24 (m, 12H), 0.87 (t, 3H, J=6.75 Hz); ¹³C NMR (75 MHz, CDCl₃): δ 81.0, 73.6, 72.5, 71.4, 34.1, 32.4, 30.1, 29.8, 26.2, 24.7, 23.3, 14.7; IR (neat) IR (neat): ν 3392.1, 2924.1, 2855.1, 2362.0, 1653.2, 1457.1 cm⁻¹; HRMS: calculated 198.30, found 181.2777 for [M−H₂O+H]⁺.

C. Synthesis of Panaxytriol

CuCl (1.5 mg), NH₂OH.HCl (10 mg) and ethylamine (0.23 mL) were added to a methanol solution of the terminal alkyne 5 (41 mg, 0.207 mmol) at room temperature. A dichloromethane solution of the alkynyl bromide 6 (24.4 mg, 0.151 mmol) was added dropwise to the reaction mixture at 0° C. over 1 hour using a syringe pump. For an additional 1 hour, the reaction mixture was stirred at 0° C. The reaction mixture was quenched with water, extracted with dichloromethane, washed with brine, dried over MgSO₄, and concentrated under reduced pressure. The residue was purified using flash column chromatography (hexane/ethyl acetate 2:1) to provide panaxytriol (38.7 mg, 92% isolated): R_{f}: 0.13 (hexane/ethyl acetate 2:1); [α]_{D}^{25°}: −21.8 (c=0.8, CHCl₃); ¹H NMR (400 MHz, CDCl₃): δ 5.94 (ddd, 1H, J=17.0, 10.1, 5.35 Hz), 5.47 (ddd, 1H, J=17.0, 1.31, 1.21 Hz), 5.25 (ddd, 1H, J=10.4, 1.25, 1.15 Hz), 4.92 (d, 1H, J=5.35 Hz), 3.62 (m, 2H), 2.58 (d, 2H, J=5.78 Hz), 2.11 (br s, 3H), 1.51-1.25 (m, 12H), 0.88 (t, 3H, J=6.73 Hz); ¹³C NMR (75 MHz, CDCl₃): δ 36.4, 117.6, 78.5, 75.1, 73.5, 72.5, 71.3, 66.9, 63.9, 34.0, 32.2, 29.9, 29.6, 26.0, 25.4, 23.0, 14.5; IR (neat): ν 3524.8, 2930.7, 2854.8, 2360.0, 1457.1 cm⁻¹; HRMS: calculated 278.39, found 261.1047 for [M−H₂O+H]⁺.

### 6.2 Example 2

### Synthesis of Compound (A)

To a THF solution of panaxytriol (0.61 g, 2.191 mmol) were added Me₂C(OCH₃)₂ (3 mL, 21.91 mmol) and p-TsOH (42 mg, 0.2191 mmol) at room temperature. After stirring overnight, the reaction mixture was quenched with saturated NaHCO₃. After an aqueous workup, the resultant mixture was purified using flash column chromatography (hexane/ethyl acetate 15:1 to 7:1) to provide Compound (A) (0.6567 g, 94%) as a colorless oil: R_{f}:0.19 (hexane:ethyl acetate=8:1); [α]_{D}^{25.7°}: +5.0 (c=0.47, acetone); ¹H NMR (400 MHz, CDCl₃): δ 5.95 (ddd, 1H, J=17.0, 10.1, 5.3 Hz), 5.46 (d, 1H, J=17.0 Hz), 5.25 (d, 1H, J=10.1 Hz), 4.91 (d, 1H, J=5.3 Hz), 3.80 (dt, 1H, J=7.7, 4.2 Hz), 3.72 (dt, 1H, J=7.9, 5.3 Hz), 2.60 (m, 2H), 1.2-1.7 (m, 12H), 1.37 (s, 6H), 0.89 (t, 3H, J=6.8 Hz); ¹³C NMR (75 MHz, CDCl₃): δ 136.3, 117.6, 109.1, 80.9, 78.6, 77.2, 75.0, 71.5, 66.9, 64.0, 33.5, 32.4, 30.3, 29.8, 28.0, 27.7, 26.6, 24.2, 23.3, 14.8; IR (neat): ν 3434.8, 2927.4, 2856.3, 2256.2, 1716.7, 1458.2, 1377.4, 1242.1, 1220.6, 1066.2, 985.9, 930.6 cm⁻¹; HRMS: calculated for [M-CH₃—H] 303.1960, found 303.1946.

### 6.3 Example 3

### Synthesis of Compound (B)

To a THF solution of panaxytriol (6 mg, 0.02155 mmol) was added MnO₂ (22 mg, 0.251 mmol) at room temperature. After stirring overnight, the reaction mixture was filtered through a short column of Celite and the solvent was removed. The concentrated reaction mixture was purified using flash column chromatography (hexane/ethyl acetate 4:1 to 2:1) to provide the Compound (B) (4.5 mg, 76%) as a colorless oil: R_{f}: 0.19 (hexane:ethyl acetate=3:1); [α]_{D}^{20.7°}: +14.4 (c=0.44, CHCl₃); ¹H NMR (400 MHz, CDCl₃): δ 6.55 (d, 1H, J=17.3 Hz), 6.41 (dd, 1H, J=17.3, 10.0 Hz), 6.22 (d, 1H, J=10.0 Hz), 3.72 (m, 1H), 3.61 (m, 1H), 2.68 (d, 2H, J=6.2 Hz), 1.2-1.6 (m, 12H), 0.88 (t, 3H, J=6.6 Hz); ¹³C NMR (75 MHz, CDCl₃): δ 178.1, 138.1, 134.8, 86.5, 77.6, 73.4, 72.3, 71.2, 66.1, 34.0, 32.3, 29.9, 29.6, 25.9, 25.7, 23.0, 14.9; IR (neat): ν 3300.3, 2945.4, 2850.4, 2231.9, 2150.6, 1650.8, 1607.1, 1463.4, 1400.9, 1257.2, 1163.5, 1132.3, 1094.8, 1026.0, 976.1, 938.6, 788.6 cm−¹; HRMS: calculated for [M+H] 277.1804, found 277.1808.

### 6.4 Example 4

### Synthesis of Compound (C)

To a THF solution of Compound (A) (29.9 mg, 0.09389 mmol) was added MnO₂ (81.6 mg, 0.9389 mmol) at room temperature. After stirring overnight, the reaction mixture was filtered through a short column of Celite and solvent was removed. The concentrated reaction mixture was purified using flash column chromatography (hexane/ethyl acetate 4:1 to 2:1) to provide the Compound (C) (18.4 mg, 62%) as a colorless oil: R_{f} 0.48 (hexane:ethyl acetate=8:1); [α]_{D}^{22.6°}: +8.6 (c=0.5, CHCl₃); ¹H NMR (400 MHz, CDCl₃): δ 6.57 (d, 1H, J=17.3 Hz), 6.41 (dd, 1H, J=17.4, 10.0 Hz), 6.22 (d, 1H, J=10.0 Hz), 3.77 (m, 2H), 2.69 (m, 2H), 1.59 (m, 2H), 1.41 (s, 6H), 1.26-1.40 (m, 10H), 0.88 (t, 3H, J=6.6 Hz); ¹³C NMR (75 MHz, CDCl₃): δ 178.1, 138.2, 134.7, 109.3, 85.6, 80.7, 78.2, 71.2, 66.2, 33.2, 32.2, 30.0, 29.5, 27.8, 27.4, 26.3, 24.2, 23.0, 14.5; IR (neat): ν 2985.9, 2929.1, 2857.5, 2236.0, 2153.0, 1734.0, 1717.0, 1645.5, 1616.4, 1456.5, 1379.2, 1290.0, 1243.0, 1163.6, 1070.0, 980.0, 789.3 cm⁻¹; HRMS: calculated for [M+H] 317.2117, found 317.2123.

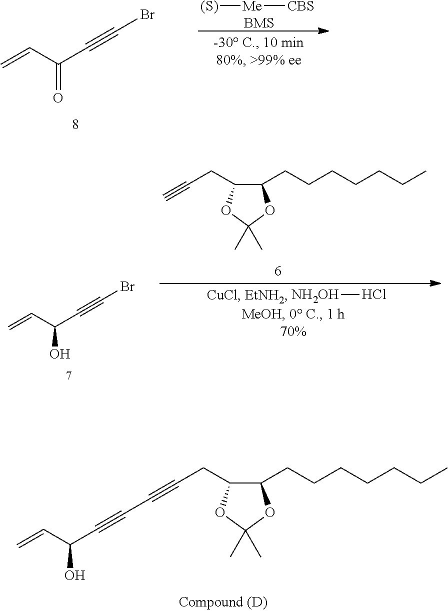

### Synthesis of Compound (D)

(S)-Me-CBS reagent (2.14 mL, 2.14 mmol, 1.0 M in toluene solution) was transferred into a freshly flame-dried flask, and the toluene was completely removed in vacuo over 1 day. The (S)-Me-CBS reagent was diluted with THF, the resulting solution was transferred to a flask containing compound 8 (160 mg, 1.05 mmol) at room temperature, and the reaction was cooled to −30 ° C. At −30° C., BH₃-Me₂S (BMS) (0.60 mL, 1.2 mmol) was added slowly over 15 minutes. After addition of BMS, thin-layer chromatography (TLC) analyses indicated the reaction was complete. Methanol was slowly added and the reaction mixture was slowly warmed to room temperature. The reaction mixture was diluted with diethyl ether, washed with 2:1 (v:v) NaOH/sat. NaHCO₃ solution until the aqueous phase was clear, and then washed with brine. After being dried over MgSO₄, the solvent was removed. The crude material was purified by silica-gel column chromatography to afford the desired product 7 (see Scheme above) (130 mg, 80%, >99% ee) as a colorless oil. CuCl (2.0 mg), NH₂OH—HCl (10.0 mg) and ethylamine (0.23 mL) were added to a methanol solution of acetonide compound 6 (see Scheme above) (45 mg, 0.205 mmol) at room temperature. The acetonide 6 can be made by converting Compound 5 to an acetonide under conditions disclosed for making Compound (A) in Example 2. A methylene chloride solution of compound 7 (25 mg, 0.152 mmol) was added dropwise to the reaction mixture at 0° C. over 1 hour. The reaction mixture was stirred at 0° C. for an additional hour. The reaction mixture was quenched with water and extracted with methylene chloride. The methylene chloride extract was washed with brine, dried over MgSO₄, and concentrated in vacuo. The residue was purified by silica-gel column chromatography to afford compound (D) (36 mg, 80%) as a colorless oil. ¹H NMR (400 MHz, CDCl₃): δ 5.90 (ddd, 1H, J=17.0, 10.0, 5.6 Hz), 5.41 (d, 1H, J=17.0 Hz), 5.20 (d, 1H, J=10.0 Hz), 4.86 (d, 1H, J=5.6 Hz), 3.82 (dt, 1H, J=7.8, 4.0 Hz), 3.65 (dt, 1H, J=8.0, 5.2 Hz), 2.59 (m, 2H), 1.2-1.7 (m, 12H), 1.36 (s, 6H), 0.90 (m, 3H). ¹³C NMR (100 MHz, CDCl₃): δ 136.0, 117.2, 108.5, 80.4, 78.1, 76.7, 75.1, 72.0, 66.4, 64.0, 33.2, 21.8, 30.2, 29.4, 28.0, 27.5, 26.3, 24.1, 23.0, 14.6. MS (EI+) calcd for [M+H] C₂₀H₃₁O₃: 319.2274; found 319.2268.

### 6.6 Example 6

### Synthesis of Compound (E)

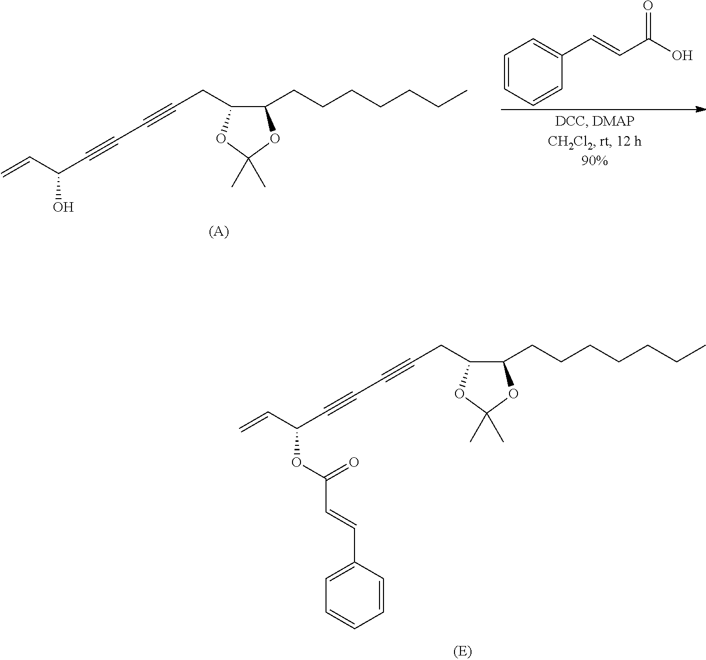

Compound (A) (23 mg, 0.072 mmol) was dissolved in 1.0 mL of anhydrous methylene chloride. To this solution was added trans-cinnamic acid (21 mg, 0.144 mmol), DCC (28 mg, 0.159 mmol) and DMAP (28 mg, 0.281 mmol). The reaction mixture was stirred at room temperature for 12 hours. The mixture was filtered and washed with methylene chloride. The solution was concentrated in vacuo, and the residue was purified by silica-gel column chromatography using hexane:ethyl acetate (10:1) to afford the ester (E) (29 mg, 90%) as a colorless oil. ¹H NMR (400 MHz, CDCl₃): δ 7.72 (d, 1H, J=16.0 Hz), 7.52 (m, 2H), 7.37 (m, 3H), 6.42 (d, 1H, J=16.0 Hz), 6.04 (d, 1H, J=5.6 Hz), 5.92 (ddd, 1H, J=17.0, 10.0, 5.6 Hz), 5.58 (d, 1H, J=17.0 Hz), 5.34 (d, 1H, J=10.0 Hz), 3.77 (dt, 1H, J=7.8, 4.0 Hz), 3.70 (dt, 1H, J=8.0, 5.2 Hz), 2.589 (m, 2H), 1.2-1.7 (m, 12H), 1.38 (s, 6H), 0.86 (m, 3H). MS (EI+) calcd for [M] C₂₉H₃₆O₄: 448.2614; found 319.2268.

### 6.7 Example 7

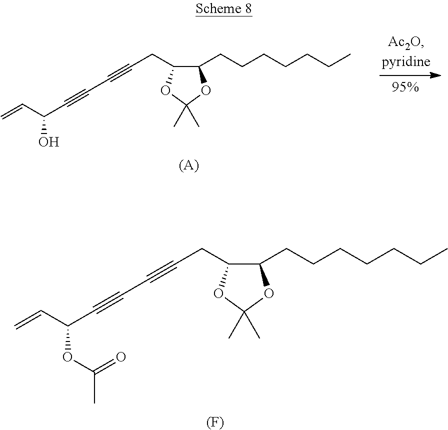

Compound (A) (10 mg, 0.031 mmol) was dissolved in 0.2 mL of anhydrous pyridine. To this solution was added 0.1 mL of acetic anhydride. The reaction mixture was stirred at room temperature for 2 hours. The mixture was quenched with sat. NaHCO₃, extracted with ethyl acetate, washed with brine and dried over MgSO₄. The solution was filtered, concentrated in vacuo, and the residue was purified by silica-gel column chromatography using hexane:ethyl acetate (10:1) to afford the acetate (F) (9 mg, 90%) as a colorless oil.

### 6.8 Example 8

### Determination of the In vitro Cytotoxicity of Panaxytriol Compounds Against Tumor Cell Lines

CCRF-CEM human T-cell acute lymphoblastic leukemia cells and the corresponding vinblastine-resistant cells (CCRF-CEM/VBL100) were cultured at an initial density of 5×10⁴ cells per milliliter. The cultured cells were then maintained in a 5% CO₂-humidified atmosphere at 37° C. in RPMI medium 1640 (GIBCO/BRL) containing penicillin (100 units/mL), streptomycin (100 μg/mL, GIBCO/BRL), and 10% heat-inactivated fetal bovine serum. The cytotoxicity of each Panaxytriol Compound was measured using the 2,3-bis(2-methoxy-4-nitro-5-sulfophenyl)-5-[(phenylamino)carbonyl]-2H-tetrazolium hydroxide (XTT) microculture method as described in Scudiero et al., Cancer Res., 48:4827 (1988). Results of the XXT assay following 72-hour inhibition are shown in Tables 3, 3A, and 3B, where VBL means vinblastine resistant cells. All compounds tested exhibited anti-cancer activity in the assay.

TABLE 3

IC₅₀ (μM)

| Compound | CCRF-CEM | CCRF ± CEM/VBL^{a} |
|---|---|---|
| panaxytriol | 12.09 ± 1.57 | 14.56 ± 6.92 (1.20x) |
| Compound A | 1.98 ± 0.20 | 2.41 ± 0.05 (1.22x) |
| Compound B | 4.49 ± 0.41 | 6.29 ± 1.41 (1.40x) |
| Compound C | 3.23 ± 0.78 | 3.84 ± 0.08 (1.19x) |

^{a}Resistance to Vinblastine was 400-fold as indicated by IC₅₀ increases (0.0020 ± 0.0007 μM in CCRF-CEM compared to 0.80 ± 0.10 μM in CCRF-CEM/VBL cell line).

A comparison of the in vitro cytotoxicity of Compounds (D) and (A) in a cancer cell assay was undertaken using the above assay procedure. The results are disclosed in Table 3A, and following.

TABLE 3A
Comparision of cytotoxicity of Compound (D) with
Compound (A) in vitro^{a}

| | | IC₅₀ (μM) | |
|---|---|---|---|
| | | CCRF- | CCRF- |
| Compound | CCRF-CEM | CEM/Taxol^{b} | CEM/VBL^{b} |
| (A) | 0.803 | Not Done | 1.122 |
| | | | [1.4-fold |
| | | | resistance]^{c} |
| | 6.150^{d} | 17.23^{d} | 6.816^{d} |
| (D) | 0.478 | 1.035 | 0.947 |
| | (New analog is | [2.17-fold | [1.98-fold |
| | 1.68-fold more potent) | resistance]^{c} | resistance]^{c} |
| | | | (New analog is |
| | | | 1.19-fold more |
| | | | potent) |

^{a}Cell growth inhibition was measured by the above assay using a Powerwave XS spectrophotometer. IC₅₀ values were determined in duplicate or triplicate from the dose-effect relationship at six or seven concentrations of each drug using the CompuSyn software by Chou and Martin, discussed below, based on the median-effect principle and plot and serial deletion analysis.
^{b}CCRF-CEM/Taxol and CCRF-CEM/VBL are subcell lines of CCRF-CEM cells that are 283-fold resistant to Taxol, and 261-fold resistant to Vinblastine, respectively, when comparing with the IC₅₀ of the CCRF-CEM cell line.
^{c}Numbers in the brackets correspond to the amount of resistance against the tested compound in the drug-resistant cell line, which was determined by comparing the IC₅₀s observed in the corresponding assay using the parent, non-drug-resistant cell line to that observed in the drug-resistant cell line. The difference is expressed mathematically by the fold increase in the IC₅₀ observed in the assay using the drug-resistant cell line.
^{d}These assays were conducted using Compound (A) that had been stored in DMSO at below approximately −20° C. for 4 years and 4 months.

The experimental data shown in Table 3A show that Compound (D) is about 1.44-fold more potent than Compound (A) in vitro, and that both Compounds (A) and (D) are active against cancer, as shown by activity against illustrative cancer cell lines, and against illustrative taxol or vinblastine-resistant (VBL) cell lines.

The cytotoxicity of Compound (E) was studied using the procedures discussed above for Table 3A. The result is disclosed in Table 3B, which indicates that Compound (E) is also active against cancer as shown by its activity against the illustrative cancer-cell line, CCRF-CEM.

TABLE 3B
Cytotoxic Potency of Compound (E)
against CCRF-CEM cell line growth in vitro^{a}

| | IC₅₀ (in μM) |
|---|---|
| | for human leukemic lymphoblastic leukemia cells |
| Compound | CCRF-CEM |
| (E) | 6.512 |

### 6.9 Example 9

### Efficacy of Panaxytriol and Compound (A)

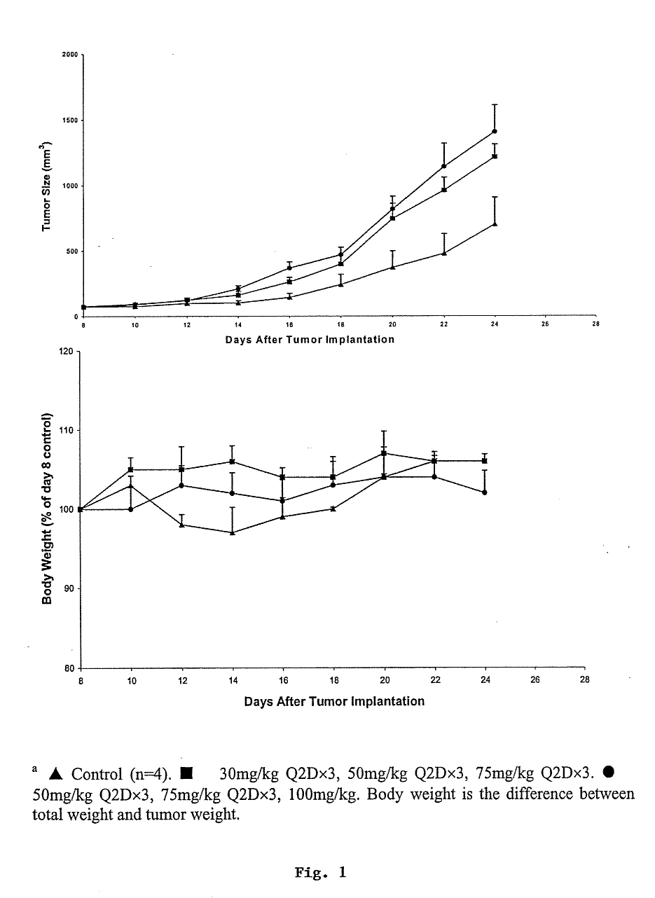

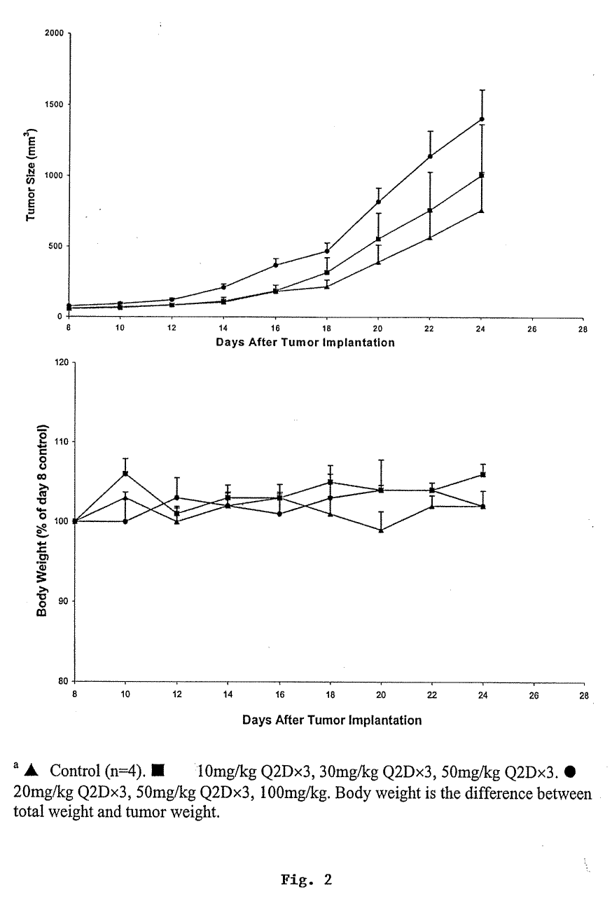

Nude mice having human mammary carcinoma xenograft MX-1 were treated with panaxytriol or Compound (A) at various dosages through the slow i.v. infusion protocol by Chou et al. (Chou, T. C., et al, Proc. Natl. Acad. Sci. U.S.A. 95: 15798 (1998)). Mice treated with 30 mg/kg of panaxytriol exhibited some suppression of tumor growth, but no significant reduction in tumor mass was observed (FIG. 1). At elevated dosage levels, improved inhibitory effects were observed. Compound (A) demonstrated enhanced in vivo potency, inhibiting tumor growth at levels as low as 10 mg/kg (FIG. 2). Elevated dosages led to enhanced tumor-growth suppression, although treatment with Compound (A) did not lead to a reduction in the tumor mass. Notably, even at the highest dosage levels (100 mg/kg), no body weight decrease was observed upon treatment with either panaxytriol or Compound (A).

### 6.10 Example 10

### Determination of the In Vivo Cytotoxicity of a Panaxytriol Compound and a Tubulin-Binding Drug

Using male athymic nude mice bearing the nu/nu gene (6 weeks of age or older, weighing between 20 and 22 g, obtainable from NCI, Frederick, Md.) into which one or more human tumor xenografts have been implanted, the in vivo cytotoxicity of a Panaxytriol Compound and a tubulin-binding drug can be determined according to the procedure set forth in Chou et al., Proc. Natl. Acad. Sci. U.S.A. 95:15798 (1998).

### 6.11 Example 11

### In-Vitro Efficacy of a Combination of a Panaxytriol Compound and a Tubulin-Binding Drug

Efficacy of panaxytriol in combination with Fludelone was measured according to procedures described in more detail by Chou et al. (1984) Adv. Enz. Regul., 22:27-55, Chou et al. (1994) Nat'l Cancer Inst., 86:1517-1524, and Chou (2006) Pharmacological Reviews, 68:621-681. Software packages used for data analyses were CalcuSyn for Windows (Chou et al., Multiple-drug dose effect analyzer and manual, Biosoft, Cambridge Place, Cambridge, U.K (1996)) and CompuSyn for Drug Combinations (Chou et al., Software for determination of synergism and antagonism and determination of IC₅₀, ED₅₀ and LD₅₀, ComboSyn Inc., Paramus, N.J. (2005)). The results of the combination of panaxytriol and Fludelone against human mammary carcinoma MX-1 cells growth in vitro^{a,e} are shown in Tables 4A, 4B and 4C.

TABLE 4A

| | Dose-effect parameters^{b} | | |
|---|---|---|---|
| Compound | m | Dm(IC₅₀) | r |
| Panaxytriol | 2.600 | 3.191 μM | 0.993 |
| Fludelone | 0.619 | 0.0027 μM | 0.992 |
| Panaxytriol | | 1.199 μM | |
| + | 1.697 | + | 0.984 |
| Fludelone | | 0.0012 μM | |

^{a}XTT/CCK-8 assays were carried out using 6 to 8 concentrations of each drug and their combinations in duplicate. Drug exposure time was 72 hrs. Absorbance was measured by using a microplate reader.
^{b}The m values signify shapes: m = 1, >1 and <1 represent hyperbolic, sigmoidal, and flat sigmoidal, respectively. The Dm values signify potency, i.e., the IC₅₀ values. The r values are the linear correlation coefficients of the dose-effect plot, which signify the conformity to the mass-action law principle.
^{e}The values of Dm, r, CI and DRI were calculated using a computer software, CompuSyn, by Chou and Martin (ComboSyn, NJ, 2005).

TABLE 4B

Combination Index^{c} (CI) at

| Compound | IC₅₀ | IC₇₅ | IC₉₀ | IC₉₅ |
|---|---|---|---|---|
| Panaxytriol | | | | |
| + | 0.816 | 0.613 | 0.635 | 0.708 |
| Fludelone | | (Synergism) | | |

^{c}CI = 1, <1, and >1 indicates additive effect, synergism, and antagonism, respectively.

TABLE 4C

Dose-Reduction Index^{d}
(DRI) at

| Compound | IC₅₀ | IC₇₅ | IC₉₀ | IC₉₅ |
|---|---|---|---|---|
| Panaxytriol | 2.66 | 2.12 | 1.70 | 1.46 |
| + | | | | |
| Fludelone | 2.27 | 7.02 | 21.69 | 46.73 |

^{d}DRI represents how may fold dose reduction is allowed at a given effect level as a result of synergistic interaction of two compounds.

A similar study with the combination of two tubulin binding-drugs (Fludelone and taxol) showed no synergy to only moderate synergy. A comparison of the results of studies with the combination of Fludelone and Panaxytriol and the combination of Fludelone and taxol is shown in Table 5.

TABLE 5

| Drug combination | At IC₉₀ & IC₉₅ | At IC₇₅ | At IC₅₀ |
|---|---|---|---|
| Fludelone + | Synergism | Synergism | Synergism |
| panaxytriol | | | |
| Fludelone + taxol | Moderate | Slight Synergism | Additive effect |
| | Synergism | | |

The results in Table 5 indicate that panaxytriol, an illustrative Panaxytriol Compound, in combination with fludelone, an illustrative tubulin-binding drug, exhibits synergism, while a combination of two tubulin-binding drugs exhibits only an additive effect to moderate synergism.

### 6.12 Example 12

### In-Vitro Neurotrophic Activity of Panaxytriol

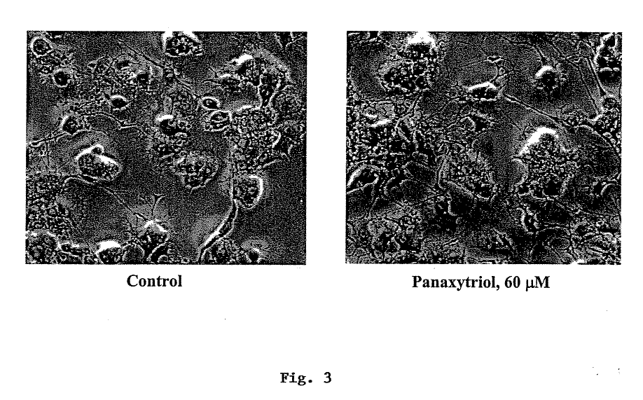

Rat pheochromocytoma cells (PC12 cells) were treated with 50 ng/mL of nerve growth factor (NGF) and with 60 μM panaxytriol for 96 hr. The cells were then compared with a similarly prepared control, lacking panaxytriol. As shown in FIG. 3, although no neurite growth was observed in the absence of panaxytriol, the sample treated with 60 μM panaxytriol demonstrated significant neurite outgrowth. This finding confirms that panaxytriol is useful for treating or preventing a neurotrophic disorder.

The present invention is not to be limited in scope by the specific embodiments disclosed in the examples. A number of references have been cited, the entire disclosures of which have been incorporated herein in their entirety.

What is claimed is:

1. A method for treating cancer or a neurotrophic disorder, comprising administering to a subject in need thereof effective amount of:
(a) a tubulin-binding drug; and
(b) panaxytriol,

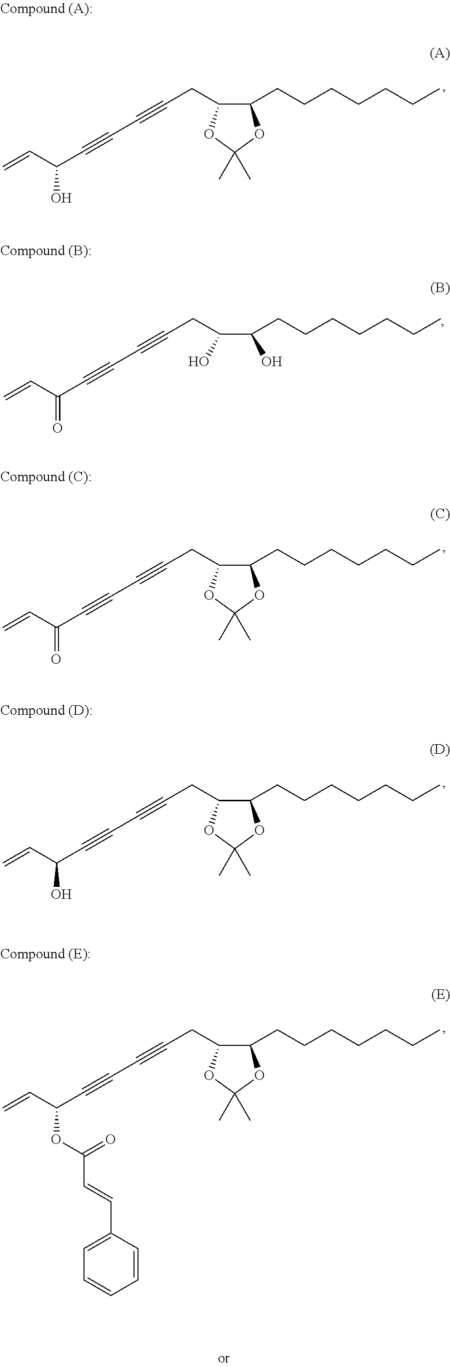

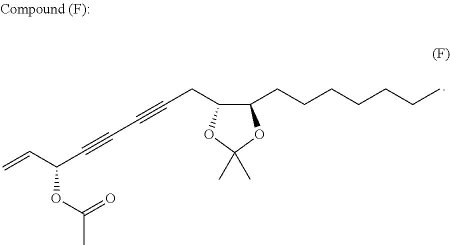

2. The method of claim 1, wherein the tubulin-binding drug is allocolchicine, amphethinile, chelidonine, colchicide, colchicine, combrestatin A1, combretastin A4, combretastain A4 phosphate, combrestatin 3, combrestatin 4, cryptophycin, curacin A, deo-dolastatin 10, desoxyepothilone A, desoxyepothilone B, dihydroxy-pentamethoxyflananone, docetaxel, dolastatin 10, dolastatin 15, epidophyllotoxin, epothilone A, epothilone B, epothilone C, epothilone D, etoposide, fludelone, griseofulvin, halichondrin B, isocolchicine, lavendustin A, methyl-3,5-diiodo-4-(4′-methoxyphenoxy)benzoate, N-acetylcolchinol, N-acetylcolchinol-O-phosphate, N-[2-[(4-hydroxyphenyl)amino]-3-pyridyl]-4-methoxybenzenesulfonamide, nocodazole, paclitaxel, phenstatin, phenylhistin, piceid, podophyllotoxin, resveratrol, rhizoxin, sanguinarine, spongistatin 1, steganacin, taxol, teniposide, thiocolchicine, vincristine, vinblastine, welwistatin, (Z)-2-methoxy-5-[2-(3,4,5-trimethoxyphenyl)vinyl]phenylamine, (Z)-3,5,4′-trimethoxystilbene (R3), 2-aryl-1,8-naphthyridin-4(1H)-one, 2-(4′-methoxyphenyl)-3-(3′,4′,5′-trimethoxybenzoyl)-6-methoxybenzo[b]thiophene, 2-methoxy estradiol, 2-strylquinazolin-4(3H)-one, 5,6-dihydroindolo(2,1-a)isoquinoline, or 10-deacetylbaccatin III.

3. The method of claim 1, further comprising administering an effective amount of another anti-cancer agent.

4. The method of claim 1, wherein the tubulin-binding drug is administered subsequent to administering panaxytriol, Compound (A), Compound (B), Compound (C), Compound (D), Compound (E), or Compound (F).

5. The method of claim 1, wherein the tubulin-binding drug is administered prior to administering panaxytriol, Compound (A), Compound (B), Compound (C), Compound (D), Compound (E), or Compound (F).

6. The method of claim 1, wherein the tubulin-binding drug is administered concurrently with panaxytriol, Compound (A), Compound (B), Compound (C), Compound (D), Compound (E), or Compound (F).

7. The method of claim 1, wherein the cancer is lung cancer, breast cancer, colorectal cancer, prostate cancer, a leukemia, a lymphoma, non-Hodgkin's lymphoma, skin cancer, a brain cancer, a cancer of the central nervous system, ovarian cancer, uterine cancer, stomach cancer, pancreatic cancer, esophageal cancer, kidney cancer, liver cancer, or a head and neck cancer.

8. The method of claim 1, wherein the panaxytriol, Compound (A), Compound (B), Compound (C), Compound (D), Compound (E), or Compound (F) is in isolated and purified form.

9. The method of claim 1, wherein the panaxytriol is naturally occurring.

10. The method of claim 1, wherein the panaxytriol, Compound (A), Compound (B), Compound (C), Compound (D), Compound (E), or Compound (F) is synthetic.

11. The method of claim 9, wherein the panaxytriol is derived from a member of the Panax genus.

12. The method of claim 11, wherein the member is Panax ginseng, Panax quinquefolius L., Panax japonicus, Panax notoginseng, Panax trifolius L., Pana vietnamensis, or Panax pseudoginseng.

13. The method of claim 11, wherein the panaxytriol is derived from the root of a member of the Panax genus.

14. The method of claim 13, wherein the member is Panax ginseng, Panax quinquefolius L., Panax japonicus, Panax notoginseng, Panax trifolius L., Pana vietnamensis, or Panax pseudoginseng.

15. The method of claim 13, wherein the panaxytriol is derived from the juice of the root.

16. A composition comprising a physiologically acceptable vehicle and an effective amount of:
(a) a tubulin-binding drug; and
(b) panaxytriol,

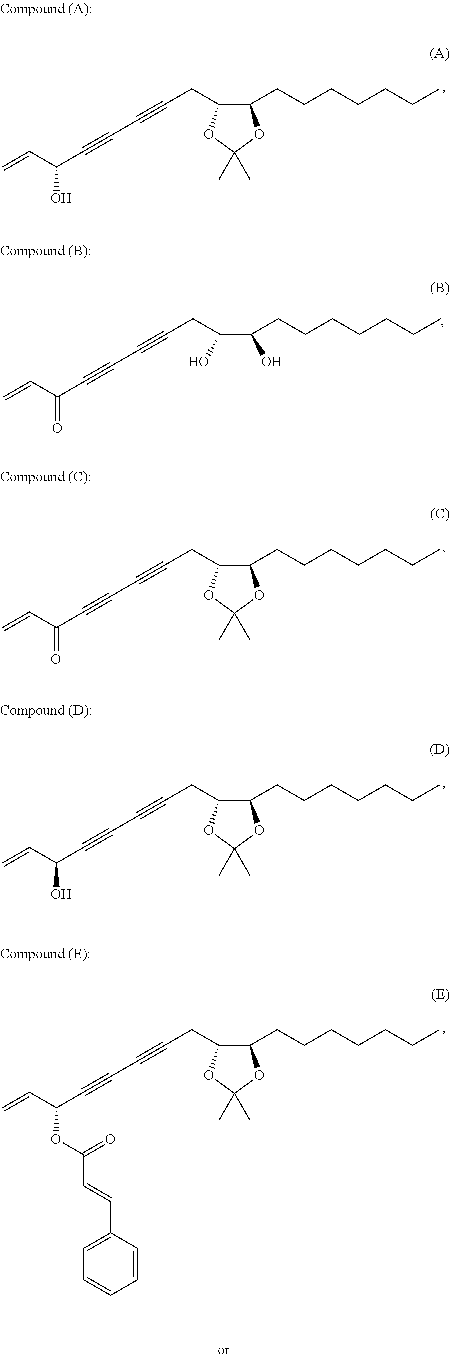

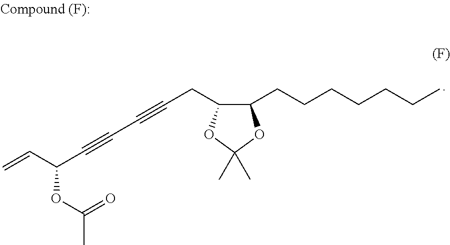

17. The composition of claim 16, wherein the tubulin-binding drug is allocolchicine, amphethinile, chelidonine, colchicide, colchicine, combrestatin A1, combretastin A4, combretastain A4 phosphate, combrestatin 3, combrestatin 4, cryptophycin, curacin A, deo-dolastatin 10, desoxyepothilone A, desoxyepothilone B, dihydroxy-pentamethoxyflananone, docetaxel, dolastatin 10, dolastatin 15, epidophyllotoxin, epothilone A, epothilone B, epothilone C, epothilone D, etoposide, fludelone, griseofulvin, halichondrin B, isocolchicine, lavendustin A, methyl-3,5-diiodo-4-(4′-methoxyphenoxy)benzoate, N-acetylcolchinol, N-acetylcolchinol-O-phosphate, N-[2-[(4-hydroxyphenyl)amino]-3-pyridyl]-4-methoxybenzenesulfonamide, nocodazole, paclitaxel, phenstatin, phenylhistin, piceid, podophyllotoxin, resveratrol, rhizoxin, sanguinarine, spongistatin 1, steganacin, taxol, teniposide, thiocolchicine, vincristine, vinblastine, welwistatin, (Z)-2-methoxy-5-[2-(3,4,5-trimethoxyphenyl)vinyl]phenylamine, (Z)-3,5,4′-trimethoxystilbene (R3), 2-aryl-1,8-naphthyridin-4(1H)-one, 2-(4′-methoxyphenyl)-3-(3′,4′,5′-trimethoxybenzoyl)-6-methoxybenzo[b]thiophene, 2-methoxy estradiol, 2-strylquinazolin-4(3H)-one, 5,6-dihydroindolo(2,1-a)isoquinoline, or 10-deacetylbaccatin III.

18. The composition of claim 16, further comprising an effective amount of another anticancer agent.

19. The composition of claim 16, wherein the panaxytriol, Compound (A), Compound (B), Compound (C), Compound (D), Compound (E), or Compound (F) is in isolated and purified form.

20. The composition of claim 16, wherein the panaxytriol is naturally occurring.

21. The composition of claim 16, wherein the panaxytriol, Compound (A), Compound (B), Compound (C), Compound (D), Compound (E), or Compound (F) is synthetic.

22. The composition of claim 20, wherein the panaxytriol is derived from a member of the Panax genus.

23. The composition of claim 22, wherein the member is Panax ginseng, Panax quinquefolius L., Panax japonicus, Panax notoginseng, Panax trifolius L., Pana vietnamensis, or Panax pseudoginseng.

24. The composition of claim 22, wherein the panaxytriol is derived from the root of a member of the Panax genus.

25. The method of claim 24, wherein the member is Panax ginseng, Panax quinquefolius L., Panax japonicus, Panax notoginseng, Panax trifolius L., Pana vietnamensis, or Panax pseudoginseng.

26. The method of claim 24, wherein the panaxytriol is derived from the juice of the root.

27-99. (canceled)

100. A method for making Compound (A): 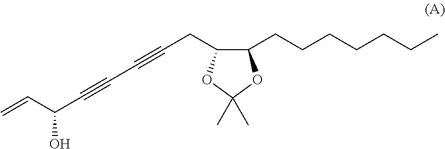
comprising allowing panaxytriol to react with 2,2-dimethoxypropane in the presence of a protic acid under conditions that are sufficient to make Compound (A).

101. A method for making Compound (B): 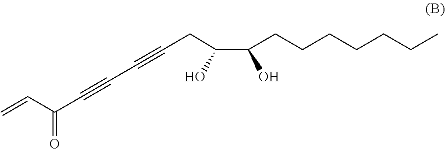
comprising oxidizing panaxytriol under conditions that are sufficient to make Compound (B).

102. A method for making Compound (C): 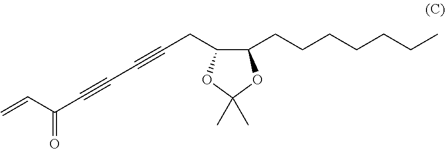
comprising oxidizing Compound (A): 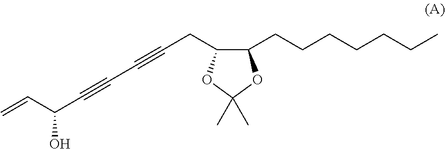
under conditions that are sufficient to make Compound (C).

103. A method for making Compound (D) 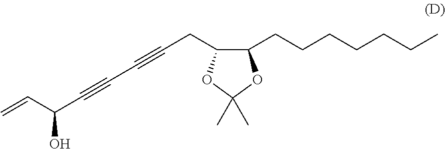
comprising allowing the compound having the structure 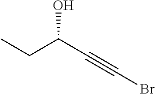
to react with the compound 6 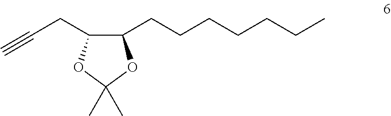
in the presence of CuCl and under conditions that are sufficient to make Compound (D).

104. A method for making Compound (E): 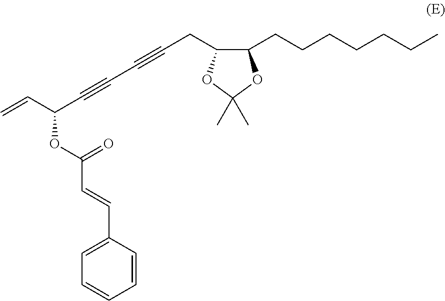
comprising allowing Compound (A) to react with cinnamic acid in the presence of a coupling agent under conditions that are sufficient to make Compound (E).

105. A method for making Compound (F): 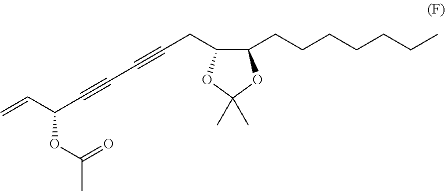
comprising allowing Compound (A) to react with acetic anhydride in the presence of a base under conditions that are sufficient to make Compound (F).

106-107. (canceled)

108. The method of claim 1, wherein the neurotrophic disorder is neutrotrophic atrophy, neurotrophic keratitis, dementia, Alzheimer's disease, amyotrophic lateral sclerosis, stroke, neuropathic pain, cancer pain, schizophrenia, Parkinson's disease, or temporal lobe epilepsy.

109-126. (canceled)

127. A compound of the structure:

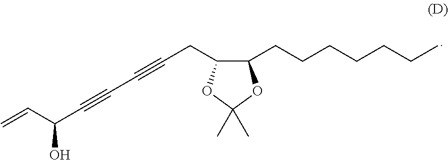

128. A compound of the structure:

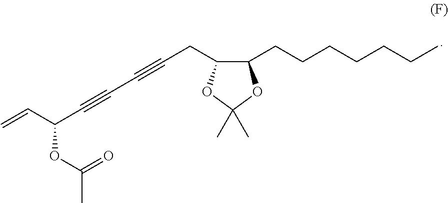

129. A compound of the structure:

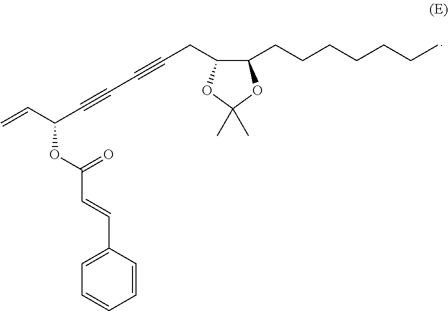

130. The composition of claim 17, wherein the tubulin-binding drug is Fludelone.
